(19) United States
(12) Patent Application Publication
(10) Pub. No.: US 2024/0254520 A1
(43) Pub. Date: Aug. 1, 2024

(54) POLYPEPTIDES AND COMPOSITIONS WITH LYTIC POLYSACCHARIDE OXIDASE ACTIVITY

(71) Applicants: INSTITUT NATIONAL DE RECHERCHE POUR L'AGRICULTURE, L'ALIMENTATION ET L'ENVIRONNEMENT, Paris (FR); IFP ENERGIES NOUVELLES, RUEIL-MALMAISON, CEDEX (FR)

(72) Inventors: Camille FILIATRAULT-CHASTEL, MARSEILLE (FR); Antoine MARGEOT, PARIS (FR); Santa BLANQUET, FOURQUEUX (FR); Jean-Guy BERRIN, ROQUEVAIRE (FR); David NAVARRO, MARSEILLE (FR)

(73) Assignees: INSTITUT NATIONAL DE RECHERCHE POUR L'AGRICULTURE, L'ALIMENTATION ET L'ENVIRONNEMENT, Paris (FR); IFP ENERGIES NOUVELLES, RUEIL-MALMAISON, CEDEX (FR)

(21) Appl. No.: 17/257,168

(22) PCT Filed: Jul. 2, 2019
(86) PCT No.: PCT/EP2019/067771

§ 371 Date: Dec. 30, 2020

(30) Foreign Application Priority Data
Jul. 2, 2018 (FR) 1856094

(51) Int. Cl. C12P 7/10; C12N 9/02

(52) U.S. Cl. CPC C12P 7/10; C12N 9/0083; C12Y 114/99

(57) ABSTRACT

The present invention in general relates to enzymes with a polysaccharide oxidase activity. In particular, the invention relates to the field of second-generation ethanol production by oxidation and enzymatic hydrolysis of materials containing polysaccharides, in particular lignocellulosic biomass. The invention further relates to the field of celluloses, and to method for producing cellulose fibres and defibrillating cellulose substrates.

### SEQUENCE LISTING

Incorporated by reference herein in its entirety is a computer-readable sequence listing submitted via EFS-Web and identified as follows: One (862 kilobyte ASCII (Text)) file named “2021_07_07_sequence_listing.txt” created on Jul. 7, 2021.

### FIELD OF THE INVENTION

The present invention relates generally to enzymes with polysaccharide oxidase activity. In particular, the invention relates to the field of second-generation ethanol production by oxidation and enzymatic hydrolysis of materials containing polysaccharides, and lignocellulosic biomass in particular.

Thus, the invention relates to the field of celluloses, as well as methods for producing cellulose fibers and for defibrillating cellulose substrates.

### TECHNOLOGICAL BACKGROUND

Lignocellulosic biomass is a polysaccharide substrate and a renewable source for producing biofuels and platform molecules for industry. Its conversion into products of interest, such as saccharides and cellulose fibers, requires the combined action of enzymes, mostly of fungal origin.

Bearing in mind the complexity and stubbornness of lignocellulosic biomass, its degradation is one of the obstacles in the development of an economically viable 2G bioethanol process.

To degrade this type of polysaccharide substrate, made up essentially of cellulose, hemicelluloses and lignin, cellulolytic microorganisms generally produce enzyme mixtures containing cellulases, hemicellulases, pectinases and lignolytic enzymes. The concerted action of various enzymes is necessary for optimal degradation of lignocellulose. In particular, the degradation of cellulose requires the coordinated and synergistic action of various types of enzymes. More particularly, efficient conversion of cellulose into small molecules requires the synergistic action of cellulases, i.e. endoglucanases (EGs), cellobiohydrolases (CBHs) and β-glucosidases. The EGs cleave the β-1,4 bonds randomly in the cellulose chains, thus liberating new terminations for the action of cellobiohydrolases (CBHs), which in their turn liberate cellobiose units. The β-glucosidases produce glucose molecules from cellobiose, thus attenuating the inhibitory effect of cellobiose on CBH.

All the enzymes with activities on carbohydrates are listed in the CAZy database (Carbohydrate Active enzymes, Lombard et al., 2014), in classes (depending on their mode of action) and in families depending on their sequence and structural parameters, which also determine the enzymatic properties.

For example, the hydrolytic enzymes are in the class of the glycoside hydrolases (GH), and the cellulases in particular are in the families GH5, GH6, GH7, GH12, GH45 and GH48.

Thus, synergistic effects have been demonstrated for enzymes with oxidative activity acting on cellulose. The first representatives of this family, the “Lytic Polysaccharide Monooxygenases” (LPMOs) were isolated from the filamentous fungi Thielavia terrestris and Thermoascus aurantiacus (Harris et al.; “Stimulation of Lignocellulosic Biomass Hydrolysis by Proteins of Glycoside Hydrolase Family 61: Structure and Function of a large, Enigmatic Family; Biochemistry (Moscl.) 49, 3305-3316; 2010). They oxidize the glycosidic bonds of the chains of polysaccharides, releasing aldonic acids, and thus increase the accessibility of the substrate for the hydrolytic enzymes.

These monooxygenases (LPMOs) are classified among the enzymes with auxiliary activity (AA) (Levasseur et al., 2014) in the families AA9, AA10, AA11, AA13, AA14 and AA15. The LPMOs with activity on cellulose belong to the family AA9. However, other families of monooxygenases have been discovered: the family AA10 includes bacterial enzymes analogous to AA9, the members of the family AA11 have activity on chitin, those of the family AA13 act on starch, those of the family AA14 act on xylan and those of the family AA15 on cellulose and chitin.

An enzyme producer often used in industry is the fungus Trichoderma reesei, which is capable of producing large amounts of enzymes. In particular, the cocktail of enzymes called “K975” represents a low diversity of enzymes and is not sufficiently effective. Comparing the genome of T. reesei against other genomes from close species, it can be seen effectively that T. reesei possesses both fewer genes involved in the degradation of the carbohydrates of the plant cell wall, and the enzymes are less diversified.

Numerous research works have therefore aimed to supplement this mixture with enzymes having higher specific activity or activity complementary to those present in T. reesei, which would make it possible to reduce the dose of enzymes to be used.

It has thus been shown that an enzyme cocktail from T. reesei containing 7% of the protein AA9 (LPMO) from T. aurantiacus is capable of hydrolyzing 90% of cellulose at 4 mg of protein per gram of cellulose, making it possible to halve the amount of enzymes required. Other members of the family AA9 have been identified in several fungal species, in particular in Podospora anserina, Myceliophthora thermophila or Phanerochaete chrysosporium.

WO 2018/050300 teaches the identification of LPMOs, and application thereof in a method for producing sugars from lignocellulosic biomass.

WO 2016/193617 teaches methods for producing nanocelluloses, comprising a step of enzymatic treatment of said substrate with a cleavage enzyme belonging to the family of the lytic polysaccharide monooxygenases (LPMOs), able to ensure oxidative cleavage of said cellulose fibers.

Despite the new pretreatment strategies, the costs of production of the nanocelluloses are still high, the yields are uncertain, and the quality and properties are variable.

Therefore there is still a need to identify new enzyme families for treating these polysaccharide substrates, and especially lignocellulosic biomass. In particular, there is still a need to identify families of enzymes capable, alone or in combination with said cocktail from T. reesei, of improving the yield from enzymatic hydrolysis.

For example, there is still a need for enzyme families capable of hydrolyzing stubborn biomasses such as miscanthus and/or poplar.

There is also still a need for enzyme families that make it possible to improve the existing methods for obtaining sugars and/or cellulose fibers starting from polysaccharide substrates such as lignocellulosic biomasses.

The invention aims to respond to these needs.

### SUMMARY OF THE INVENTION

According to a first aim, the invention relates to an isolated polypeptide with polysaccharide oxidase activity, characterized in that it comprises a sequence having an amino acid identity of at least 20% with a reference polypeptide sequence SEQ ID No. 1 or SEQ ID No. 2, using a BLAST-P comparison method, said BLAST-P comparison method giving an e-value of 1e⁻¹⁸ or less.

In particular, the invention relates to an isolated polypeptide with polysaccharide oxidase activity as defined above, characterized in that it comprises a sequence having an amino acid identity of at least 80% with a reference polypeptide sequence SEQ ID No. 1 or SEQ ID No. 2, said BLAST-P comparison method giving an e-value of 1e⁻¹⁸ or less.

Quite particularly, said isolated polypeptide with polysaccharide oxidase activity may be characterized in that it comprises a reference polypeptide sequence selected from SEQ ID No. 5 to SEQ ID No. 382 and SEQ ID No. 383.

According to a second aim, the invention relates to a composition with polysaccharide oxidase activity, characterized in that it comprises a polypeptide with polysaccharide oxidase activity as defined above.

In particular, said composition may be characterized in that it further comprises at least one polypeptide with polysaccharide degradase activity selected from: a cellulase, a hemicellulase, a ligninase and a carbohydrate oxidase.

Said composition with polysaccharide oxidase activity may be characterized in that it is obtainable from one or more organisms of the fungus or yeast type, in particular selected the from genera: Achlya, Acremonium, Aspergillus, Cephalosporium, Chrysosporium, Cochliobolus, Endothia, Fusarium, Gliocladium, Humicola, Hypocrea, Myceliophthora, Mucor, Neurospora, Penicillium, Pyricularia, Thielavia, Tolypocladium, Trichoderma, Podospora, Pycnoporus, Fusarium, Thermonospora; quite particularly Aspergillus or Trichoderma or Podospora; preferably Aspergillus or Podospora.

Said composition with polysaccharide oxidase activity may be characterized in that said polypeptide with polysaccharide oxidase activity is obtained recombinantly.

According to a third embodiment, the invention relates to a kit comprising at least:
a first part comprising a polypeptide with polysaccharide oxidase activity as defined above, or a composition comprising said polypeptide with polysaccharide oxidase activity;
a second part comprising g a polypeptide with polysaccharide degradase activity selected from a cellulase, a hemicellulase, a ligninase and a carbohydrate oxidase, or a composition comprising said polypeptide with polysaccharide degradase activity.

According to a fourth embodiment, the invention relates to a host able to express recombinantly a polypeptide with polysaccharide oxidase activity as defined above. Said host may in particular be a yeast, a bacterium or a fungus.

According to a fifth embodiment, the invention relates to an isolated nucleic acid coding for a polypeptide with polysaccharide oxidase activity as defined above.

According to a sixth embodiment, the invention relates to a method of obtaining a sugar starting from a polysaccharide substrate, comprising at least one step of contacting said substrate with a polypeptide with polysaccharide oxidase activity as defined above, or a composition with polysaccharide oxidase activity comprising said polypeptide with polysaccharide oxidase activity. Preferably, said substrate is a lignocellulosic substrate.

In particular, the invention relates to a method of producing alcohol starting from a lignocellulosic substrate, characterized in that it comprises obtaining a sugar by a method of obtaining a sugar as defined above, and the alcoholic fermentation of said sugar by an alcohol-producing microorganism.

According to a seventh embodiment, the invention relates to a method for preparing a cellulosic substrate for producing cellulose fibers, said method comprising at least the following steps consisting of:
a) providing a cellulosic substrate able to form cellulose fibers;
b) in the presence of an electron donor, contacting said substrate with at least one polypeptide with polysaccharide oxidase activity in conditions suitable for ensuring oxidative cleavage of said cellulose fibers; characterized in that said polypeptide with polysaccharide oxidase activity has an amino acid identity of at least 20% with a reference polypeptide sequence SEQ ID No. 1 or SEQ ID No. 2, using a BLAST-P comparison method, said BLAST-P comparison method giving an e-value of 1e⁻¹⁸ or less.

According to an eighth embodiment, the invention relates to a method of defibrillating a cellulosic substrate, said method comprising at least the following steps consisting of:
a) providing a cellulosic substrate able to form cellulose fibers, which had been brought into contact beforehand with an electron donor and a polypeptide with polysaccharide oxidase activity, in conditions suitable for ensuring oxidative cleavage of said cellulose fibers in the presence of an electron donor; and
b) treating said cellulosic substrate mechanically, in order to defibrillate said substrate, characterized in that said polypeptide with polysaccharide oxidase activity has an amino acid identity of at least 20% with a reference polypeptide sequence SEQ ID No. 1 or SEQ ID No. 2, using a BLAST-P comparison method, said BLAST-P comparison method giving an e-value of 1e⁻¹⁸ or less.

According to a ninth embodiment, the invention relates to a method of producing cellulose fibers, said method comprising at least the following steps consisting of:
a) providing a cellulosic substrate able to form cellulose fibers;
b) in the presence of an electron donor, contacting said cellulosic substrate with at least one polypeptide with polysaccharide oxidase activity in conditions suitable for ensuring oxidative cleavage of said cellulose fibers;
c) treating said cellulosic substrate mechanically, in order to produce said cellulose fibers starting from said cellulosic substrate,
   characterized in that said polypeptide with polysaccharide oxidase activity has an amino acid identity of at least 20% with a reference polypeptide sequence SEQ ID No. 1 or SEQ ID No. 2, using a BLAST-P comparison method, said BLAST-P comparison method giving an e-value of 1e⁻¹⁸ or less.

According to a tenth embodiment, the invention relates to cellulose fibers resulting from a defibrillation process and/or a method of producing cellulose fibers as defined above.

### DESCRIPTION OF THE FIGURES

FIG. 1: Monitoring the hydrolysis yield over time, with the cocktail from T. reesei; on three pretreated biomasses. The error bars represent the standard deviation calculated 3 for replicates. Ordinate: hydrolysis yield on three pretreated biomasses A. On straw; B. On miscanthus; C. On poplar (expressed in percentage relative to the cellulose available in the initial substrate). Abscissa: time in hours starting from the hydrolysis time 0. Curve A. On straw: Increase in hydrolysis yield up to a value of 90% at Time (h) 140. Curve B. On poplar: Increase in hydrolysis yield up to a value of 60% at Time (h) 140. Curve C. On miscanthus: Increase in hydrolysis yield up to a value of 50% at Time (h) 140.

FIG. 2A-2C: Change in hydrolysis yield in the presence of secretomes derived from the strain CIRM-BRFM 1490 of Aspergillus japonicus, relative to the hydrolysis yield in the presence of the reference cocktail alone. A. On straw B. On miscanthus C. On poplar. The mean values were calculated on 7 to 15 replicates per point (the asterisks * indicate the mean values for which the Student test relative to the reference gives a p-value <0.05). Ordinate: Improvement in hydrolysis yield, the values of which are characterized from −15% to 15%. Abscissa: the various secretomes of the strain CIRM-BRMF 1490, depending on the substrates on which they were produced (AMB: autoclaved maize bran, SBP: beet pulp, Avicel), tested in supplementation of the reference cocktail on one of the three biomasses at 24, 48 or 96 h of hydrolysis. A. On straw: The lighter boxes illustrate the performance of the secretomes on straw at 24 h (to the left of each entry) and the darker boxes illustrate the performance of the secretomes on straw at 48 h (to the right of each entry). B. On miscanthus: The lighter boxes (on the left) illustrate the performance of the secretomes on miscanthus at 24 h and the darker boxes (on the right) illustrate the performance of the secretomes on miscanthus at 96 h. C. On poplar: The lighter boxes (on the left) illustrate the performance of the secretomes on poplar at 24 h and the darker boxes (on the right) illustrate the performance of the secretomes on poplar at 96 h.

FIG. 3: Conservation of the catalytic module and consensus sequence. Graphical representation of the consensus amino acids during alignment of 378 X273 modules, generated using the WebLogo application (Crooks et al., 2004). Ordinate, unit of sequence conservation expressed in bits. Abscissa, position in the reference consensus sequence of the module. The size of the amino acids is represented as a function of the frequency observed, as defined according to the algorithm given in Crooks et al. (2004).

FIG. 4: Synergy of the X273 with CBHI for the liberation of cellobiose from cellulose nanofibrils. The enzymes AaX273 and Pax273 (8 μg) reacted with a mixture of cellulose nanofibrils (CNFs) at 0.1% for 16 h in the presence of ascorbate (1 mM), then the CNFs were washed and hydrolyzed with cellulase CBHI from T. reesei (0.8 μg) for 30 minutes. Ordinate: Area of the cellobiose peaks (in nC·min). Abscissa: (on the left) cellulase CBHI in the presence of CNF, control, (in the center) cellulase CBHI supplemented with enzyme with polysaccharide oxidase activity derived from Aspergillus aculeatus (referenced AaX273). (on the right) cellulase CBHI supplemented with enzyme with polysaccharide oxidase activity derived from Podospora anserina (referenced Pax273). The margin of error represents the standard deviation.

FIG. 5: Concentration of glucose liberated after 24 h of hydrolysis of miscanthus with the reference cocktail, with or without addition of X273. Sequential assay: the X273 (2.2 μM) acted for 24 h, then the cocktail K975+SP188 (1 mg/g DM) was added for 24 h. Simultaneous assay: the X273 and the cocktail K975+SP188 were added together for 24 h. The error bars represent the standard deviation calculated on 3 replicas. Ordinate: concentration of glucose liberated (in g/L). Abscissa: (on the left) miscanthus substrate in the presence of T. reesei cocktail, control, (in the center) in the presence of T. reesei cocktail supplemented with enzyme with polysaccharide oxidase activity derived from Aspergillus aculeatus (referenced AaX273). (on the right) in the presence of T. reesei cocktail supplemented with enzyme with polysaccharide oxidase activity derived from Podospora anserina (referenced Pax273). For each of the three experiments, two types of hydrolysis are carried out, sequential (on the left) or simultaneous (on the right).

FIG. 6: Improvement in the accessibility of cellulose by X273, by measuring the cellobiose liberated in the presence of a cellulase from Trichoderma reesei (CBH1). The test is carried out on PASC (Phosphoric Acid Swollen Cellulose), treated in the absence of enzyme X273 (left-hand column), in the presence of AaX273 derived from Aspergillus aculeatus (SEQ ID No. 2), AjX273 derived from Aspergillus japonicus (SEQ ID No. 383), PfX273 derived from Pestalotiopsis fici (SEQ ID No. 175) or ApX273 derived from Aureobasidium pullulans (SEQ ID No. 208)—from left to right. Ordinate, amount of cellobiose liberated, expressed in mg/L.

### DETAILED DESCRIPTION OF THE INVENTION

In order to overcome the aforementioned drawbacks of the prior art, the inventors endeavoured to identify secretomes of Aspergillus capable of supplementing a reference cellulolytic cocktail derived from T. reesei on a series of so-called stubborn lignocellulosic biomasses, such as straw, poplar or miscanthus.

For this purpose, the inventors produced secretomes starting from cultures in different conditions and from several strains of Aspergillus, selected from the collection of CIRM-CF (International Center for Microbial Resources—Filamentous Fungi) of INRA.

In particular, secretomes giving the greatest improvements in hydrolysis yield of miscanthus were selected.

A protein of interest appeared during exploration of the contents of the secretomes, which is only present in the secretomes capable of improving the hydrolysis of miscanthus. It had not been recognized during CAZy annotation. However, comparing its sequence with the database of the NCBI BLAST tool, and using a server for prediction of 3D structure, it was found that this protein contains a module having similarities with those found in the AA10 family, followed by a C-terminal extension predicted as a disordered region. After verification using the CAZy tools, it was then established that this protein does not belong to the AA10 family, but to a family not yet characterized called X273 (CAZy internal referencing).

Surprisingly, it was shown that this family of enzymes displays behavior of the polysaccharide oxidase type (LPMO), which is materialized in particular by the presence of a copper ion within its active center and the production of H₂O₂ in the presence of an electron donor such as ascorbic acid.

It has also been shown that, in the presence of cellulases, this enzyme family displays synergistic activity on cellulose and quite particularly on cellulose nanofibrils (CNFs).

Without wishing to be bound to any theory, the inventors are of the opinion that the X273 family (also mentioned in the description as polypeptide with polysaccharide oxidase activity according to the invention), present in the fungal genomes, is characterized by a catalytic module belonging to a new LPMO family.

According to the invention, this new family of LPMOs is therefore characterized by a polypeptide with polysaccharide oxidase activity, comprising a sequence having amino acid identity of at least 20% with a reference polypeptide sequence SEQ ID No. 1 (derived from Podospora anserina) or SEQ ID No. 2 (derived from Aspergillus aculeatus), using a BLAST-P comparison method, said BLAST-P comparison method giving an e-value of 1e⁻¹⁸ or less.

In particular, according to the invention, this new family of LPMOs is characterized by a polypeptide with polysaccharide oxidase activity, comprising a sequence having amino acid identity of at least 40% with a reference polypeptide sequence SEQ ID No. 1 (derived from Podospora anserina) or SEQ ID No. 2 (derived from Aspergillus aculeatus), using a BLAST-P comparison method, said BLAST-P comparison method giving an e-value of 1e⁻¹⁸ or less.

In particular, according to the invention, this new family of LPMOs is characterized by a polypeptide with polysaccharide oxidase activity, comprising a sequence having amino acid identity of at least 50% with a reference polypeptide sequence SEQ ID No. 1 (derived from Podospora anserina) or SEQ ID No. 2 (derived from Aspergillus aculeatus), using BLAST-P comparison method, said BLAST-P comparison method giving an e-value of 1e⁻¹⁸ or less.

In particular, according to the invention, this new family of LPMOs is characterized by a polypeptide with polysaccharide oxidase activity, comprising a sequence having amino acid identity of at least 60% with a reference polypeptide sequence SEQ ID No. 1 (derived from Podospora anserina) or SEQ ID No. 2 (derived from Aspergillus aculeatus), using a BLAST-P comparison method, said BLAST-P comparison method giving an e-value of 1e⁻¹⁸ or less.

In particular, according to the invention, this new family of LPMOs is characterized by a polypeptide with polysaccharide oxidase activity, comprising a sequence having amino acid identity of at least 70% with a reference polypeptide sequence SEQ ID No. 1 (derived from Podospora anserina) or SEQ ID No. 2 (derived from Aspergillus aculeatus), using a BLAST-P comparison method, said BLAST-P comparison method giving an e-value of 1e⁻¹⁸ or less.

In particular, according to the invention, this new family of LPMOs is characterized by a polypeptide with polysaccharide oxidase activity, comprising a sequence having amino acid identity of at least 80% with a reference polypeptide sequence SEQ ID No. 1 (derived from Podospora anserina) or SEQ ID No. 2 (derived from Aspergillus aculeatus), using a BLAST-P comparison method, said BLAST-P comparison method giving an e-value of 1e⁻¹⁸ or less.

In particular, according to the invention, this new family of LPMOs is characterized by a polypeptide with polysaccharide oxidase activity, comprising a sequence having amino acid identity of at least 90% with a reference polypeptide sequence SEQ ID No. 1 (derived from Podospora anserina) or SEQ ID No. 2 (derived from Aspergillus aculeatus), using a BLAST-P comparison method, said BLAST-P comparison method giving an e-value of 1e⁻¹⁸ or less.

The two reference sequences SEQ ID No. 1 and SEQ ID No. 2 each correspond to the complete form of the polypeptide, including (i) a signal peptide in N-terminal position, as predicted by the SignalP 4.1 software, (ii) the catalytic module including a 1st N-terminal histidine, and (iii) a C-terminal extension.

The catalytic module is responsible for the polysaccharide oxidase activity, and is characterized by the presence of an active center of the “Histidine brace” type also found in the known enzymes of the LPMO type, and capable of fixing copper.

The polypeptides with polysaccharide oxidase activity according to the invention are characterized in particular by the presence of an active site formed by two conserved histidine residues, one of the two histidine residues generally being at the N-terminal end of the catalytic module.

For this reason, the presence of the signal peptide and of the C-terminal extension is optional for obtaining polysaccharide oxidase activity.

For reference, the sequences SEQ ID No. 3 and SEQ ID No. 4 correspond respectively to the signal peptides of the sequences SEQ ID No. 1 and SEQ ID No. 2.

For reference, the sequences SEQ ID No. 5 and SEQ ID No. 6 correspond respectively to the catalytic modules of the sequences SEQ ID No. 1 and SEQ ID No. 2.

For reference, the two reference sequences SEQ ID No. 1 and SEQ ID No. 2 share 45% sequence identity between them, with an E-value of 2e⁻⁵³.

For reference, the two reference sequences SEQ ID No. 5 and SEQ ID No. 6 share 45% sequence identity between them, with an E-value of 6e⁻⁵².

In particular, 379 polypeptide sequences, all of fungal origin, have been identified (search made in May 2018 in the nr database of the NCBI BLAST-P tool): i.e. 335 sequences derived from ascomycetes, 41 sequences derived from basidiomycetes and 3 from unclassified fungi (listed as SEQ ID No. 7 to 383).

Thus, according to certain embodiments, the invention relates to an isolated polypeptide with polysaccharide oxidase activity, characterized in that it comprises a reference polypeptide sequence selected from SEQ ID No. 5 to SEQ ID No. 382 and SEQ ID No. 383.

According to one embodiment, the isolated polypeptide with polysaccharide oxidase activity according to the invention is able to bind to cellulose, and/or comprises a cellulose binding module.

According to one embodiment, the invention relates to an isolated polypeptide with polysaccharide oxidase activity, characterized in that it comprises a reference polypeptide sequence selected from SEQ ID No. 2, 34, 40, 50, 51, 60, 61, 82, 84, 86, 87, 89, 93, 97, 103, 110, 121, 146, 156, 166, 174, 179, 181, 185, 188, 189, 207, 210, 220, 227, 233, 239, 266, 270, 273, 285, 287, 291, 297, 300, 310, 319, 326.

According to one embodiment, the invention relates to an isolated polypeptide with polysaccharide oxidase activity, characterized in that it comprises a polypeptide sequence having at least 20% identity with a reference sequence selected from SEQ ID No. 2, 34, 40, 50, 51, 60, 61, 82, 84, 86, 87, 89, 93, 97, 103, 110, 121, 146, 156, 166, 174, 179, 181, 185, 188, 189, 207, 210, 220, 227, 233, 239, 266, 270, 273, 285, 287, 291, 297, 300, 310, 319, 326.

According to one embodiment, the invention relates to an isolated polypeptide with polysaccharide oxidase activity, characterized in that it comprises a polypeptide sequence having at least 40% identity with a reference sequence selected from SEQ ID No. 2, 34, 40, 50, 51, 60, 61, 82, 84, 86, 87, 89, 93, 97, 103, 110, 121, 146, 156, 166, 174, 179, 181, 185, 188, 189, 207, 210, 220, 227, 233, 239, 266, 270, 273, 285, 287, 300, 310, 319, 326.

According to one embodiment, the invention relates to an isolated polypeptide with polysaccharide oxidase activity, characterized that it a in comprises polypeptide sequence having at least 80% identity with a reference sequence selected from SEQ ID No. 2, 34, 40, 50, 51, 60, 61, 82, 84, 86, 87, 89, 93, 97, 103, 110, 121, 146, 156, 166, 174, 179, 181, 185, 188, 189, 207, 210, 220, 227, 233, 239, 266, 270, 273, 285, 287, 300, 310, 319, 326.

According to one embodiment, the invention relates to an isolated polypeptide with polysaccharide oxidase activity, characterized in that it a comprises polypeptide sequence having at least 90% identity with a reference sequence selected from SEQ ID No. 2, 34, 40, 50, 51, 60, 61, 82, 84, 86, 87, 89, 93, 97, 103, 110, 121, 146, 156, 166, 174, 179, 181, 185, 188, 189, 207, 210, 220, 227, 233, 239, 266, 270, 273, 285, 287, 300, 310, 319, 326.

Particular embodiments, using a polypeptide with polysaccharide oxidase activity according to the invention, are developed hereunder.

### GENERAL DEFINITIONS

In general, and throughout the present text, terms of the type “comprise” or “include”, as well as variations thereof, may include elements other than those explicitly mentioned. These terms may, if applicable, be replaced by “consist of”. The articles “a” and “an” also include “more than a” or “more than one”, which includes “a plurality”, or else “two or more”.

“BLAST-P method” (also called Protein Basic Local Alignment Search Tool method) denotes a method of analysis that is familiar to a person skilled in the art. The BLAST-P method is described in particular in Altschul et al. (1990, J Mol Biol, Vol. 215 (No. 3): 403-410), Altschul et al. (1997, Nucleic Acids Res. Vol. 25:3389-3402) and Altschul et al. (2005, FEBS J. Vol. 272:5101-5109). The BLAST-P method can be carried out using the NCBI tool that is available on line (internet address: http://blast.ncbi.nlm.nih.gov/Blast.cgi?PAGE=Proteins). When the BLAST-P method is used in the present application, it is preferably used according to the following parameters: (i) Expected threshold: 10; (ii) Word Size: 6; (iii) Max Matches in a Query range: 0; (iv) Matrix: BLOSSUM62; (v) Gap costs: Existence 11, Extension 1; (vi) Compositional Adjustments: Conditional compositional score matrix adjustment, (vii) No filter; (viii) No mask.

As is well known, the score of an alignment, S, is calculated as the sum of the scores for substitution and gap. The scores for substitution are given in a table (see PAM, BLOSUM below). The scores for gap are generally calculated as the sum of G, the gap opening penalty and L, the gap extension penalty. For a gap of length n, the cost of a gap would be G+Ln. The choice of the costs of the gaps, G and L are empirical, but it is usual to choose a high value for G (10-15) and a low value for L (1-2). Optimal alignment signifies alignment of two sequences with the highest possible score.

In the context of the present invention, the “percentage identity” between two polypeptides denotes the percentage of identical amino acids between the two polypeptide sequences to be compared, obtained after optimal alignment, this percentage being completely random and the differences between the two polypeptide sequences being distributed randomly over their length. Comparison of two polypeptide sequences is carried out conventionally by comparing the sequences after aligning them optimally, said comparison must be able to be carried out segment by segment or using an “alignment window”. Optimal alignment of the sequences for comparing them is performed using BLAST-P comparison software.

In principle, the percentage identity between two amino acid sequences is determined by comparing the two optimally aligned sequences, within which the nucleic acid sequences to be compared may contain additions or deletions relative to the reference sequence of the optimal alignment between the two polypeptide sequences. The percentage identity is calculated by determining the number of positions in which an amino acid is identical between the two sequences, preferably between two complete sequences, and then by dividing this number of identical positions by the total number of positions in the alignment window and multiplying the result by 100 to obtain the percentage identity between the two sequences.

As understood in the present application, polypeptide sequences having at least 20% amino acid identity with a reference sequence comprise those that have at least 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 28%, 59%, 60%, 61%, 62%, 63%, 65%, 64%, 66%, 67%, 68%, 69%, 70%, 71%, 72%, 73%, 74%, 75%, 76%, 77%, 78%, 79%, 80%, 818, 82%, 83%, 84%, 85%, 86%, 87%, 88%, 89%, 90%, 91%, 92%, 93%, 94%, 95%, 96%, 97%, 98% and 99% amino acid identity with said reference sequence.

In particular, “polypeptide sequence having at least 20% identity” means the sequences comprising at least 40% identity, quite particularly at least 80% identity, and preferably at least 90% identity with a reference sequence; such as SEQ ID No. 1 or 2.

Thus, according to certain embodiments, the invention relates to an isolated polypeptide with polysaccharide oxidase activity, characterized in that it comprises a reference polypeptide sequence having at least 40% identity with a reference sequence SEQ ID No. 5 to SEQ ID No. 383.

Thus, according to certain embodiments, the invention relates to an isolated polypeptide with polysaccharide oxidase activity, characterized in that it comprises a reference polypeptide sequence having at least 50% identity with a reference sequence SEQ ID No. 5 to SEQ ID No. 383.

Thus, according to certain embodiments, the invention relates to an isolated polypeptide with polysaccharide oxidase activity, characterized in that it comprises a reference polypeptide sequence having at least 60% identity with a reference sequence SEQ ID No. 5 to SEQ ID No. 383.

Thus, according to certain embodiments, the invention relates to an isolated polypeptide with polysaccharide oxidase activity, characterized in that it comprises a reference polypeptide sequence having at least 70% identity with a reference sequence SEQ ID No. 5 to SEQ ID No. 383.

Thus, according to certain embodiments, the invention relates to an isolated polypeptide with polysaccharide oxidase activity, characterized in that it comprises a reference polypeptide sequence having at least 80% identity with a reference sequence SEQ ID No. 5 to SEQ ID No. 383.

Thus, according to certain embodiments, the invention relates to an isolated polypeptide with polysaccharide oxidase activity, characterized in that it comprises a reference polypeptide sequence having at least 90% identity with a reference sequence SEQ ID No. 5 to SEQ ID No. 383.

Similarly, the “percentage identity” between two nucleic acid sequences represents the percentage of identical nucleotide residues between the two nucleic acid sequences to be compared, obtained after optimal alignment, this percentage being purely random and the differences between the two sequences being distributed randomly over the length of the sequences. Comparison of two nucleic acid sequences is performed conventionally by comparing the sequences after aligning them optimally, and said comparison may be carried out segment by segment or using an “alignment window”. Optimal alignment of the sequences with a view to comparing them is performed using BLAST-N comparison software.

In principle, the percentage identity between two nucleic acid sequences is determined by comparing the two optimally aligned sequences in which the nucleic acid sequences to be compared may contain additions or deletions relative to the reference sequence of the optimal alignment between the two sequences. The percentage identity is calculated according to the number of positions at which the nucleotide residues are identical between the two sequences, preferably between the two complete sequences, and then by dividing this number of identical positions by the total number of positions in the alignment window and multiplying the result by 100 to obtain the percentage identity between the two sequences.

As understood here, the nucleotide sequences having at least 20% identity with the reference sequence include those having at least least 21%, 22%, 23%, 24%, 25%, 26%, 27%, 28%, 29%, 30%, 318, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 28%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 848, 85%, 86%, 878, 88%, 89%, 90%, 91%, 92%, 93%, 94%, 95%, 96%, 97%, 98% and 99% identity of nucleotides with the reference sequence.

In particular, “nucleotide sequence having at least 20% identity” means the sequences comprising at least 40% identity, quite particularly at least 80% identity, and preferably at least 90% identity with a reference sequence; such as SEQ ID No. 1 or 2.

The BLOSUM (Blocks Substitution Matrix) matrixes are substitution score matrixes in which the scores for each position are derived from observation of the frequency of substitution of the blocks in local alignments for related proteins. Each matrix is designed for a particular distance of evolution. In the BLOSUM62 matrix, for example, the alignment from which the scores were derived was created starting from sequences not sharing more than 62% identity. The sequences that possess more than 62% identity are represented by a single sequence in the alignment so as not to over represent close members of one and the same family.

As used here, an E-value (also called Expect Value or e-value) is a parameter that is calculated when the BLAST-P method is used, said parameter representing the number of different alignments with equivalent scores or with better than S whose appearance is expected during a random search in the database. Thus, the lower the E-value, the more significant the score and the alignment will be.

Thus, an e-value of 1e⁻¹⁸ or less includes the e-values of 1e⁻²⁰ or less, 1e⁻²⁵ or less, 1e⁻³⁰ or less, 1e⁻⁴⁰ or less, 1e⁻⁵⁰ or less, 1e⁻⁶⁰ or less, 1e⁻⁷⁰ or less, 1e⁻⁸⁰ or less, 1e⁻⁹⁰ or less and 1e⁻¹⁰⁰ or less.

“Cellulosic substrate” or “lignocellulosic substrate” means in all matter of biomass (including the organic materials of vegetable origin, including algae, animal origin or fungal origin) and containing cellulose, in particular in the form of cellulosic fibers (i.e. cellulose fibers).

“Lignocellulosic biomass” means any biomass that can be used as lignocellulosic substrate. Lignocellulosic biomass may in particular be classified as a function of its origin:
agricultural residues produced during the growing of various plants intended for food;
forestry waste of hardwood or softwood, obtained from the wood industry or pulp and paper industry;
energy-producing plants derived from dedicated crops (i.e. perennial grasses and short-rotation coppice);
municipal and industrial solid waste.

For example, a lignocellulosic substrate may be obtained from a lignocellulosic biomass including: poplar, pine, miscanthus, willow, switchgrass, maize, sugar cane, wheat, rice, oat, barley, beet, olive tree, grapevine, cotton, eucalyptus, and fruit trees.

The cellulosic substrate is obtained advantageously from wood (of which cellulose is the main component), but also from any cellulose-rich fibrous plant, for example such as, cotton, flax, hemp, bamboo, kapok, coconut fiber (coir), ramie, jute, sisal, raffia, papyrus and certain reeds, sugar cane bagasse, beet (and especially beet pulp), citrus fruits, stems of maize or of sorghum, or else annual plants for straw.

The cellulose substrates may also be obtained from marine animals (such as tunicates for example), algae (for example such as Valonia or cladophora) or bacteria for bacterial cellulose (for example such as bacterial strains of the Gluconoacetobacter type).

Cellulose derived from primary walls such as the parenchyma of fruits (for example beets, citrus fruits etc.) or from secondary walls, such as wood, will be selected, depending on the applications.

The cellulosic substrate advantageously consists of a cellulosic material prepared by chemical or mechanical means, starting from any cellulose source as mentioned above (and in particular starting from wood).

Material containing lignocellulose is generally present, for example, in the stems, leaves, bran, envelopes and rachis of plants or leaves, branches, and wood from trees. In a nonlimiting manner, the material containing lignocellulose may also be herbaceous material, residues from agriculture and sylviculture, municipal solid waste, waste paper, and residues from grinding of pulp and paper. It is to be noted here that the material containing lignocellulose may be in the form of material from the cell wall of the plant containing lignin, cellulose, and hemicellulose in a mixed matrix.

According to certain particular embodiments, especially those relating to the production of cellulose fibers, the material containing lignocellulose is a lignocellulosic biomass selected from the group consisting of: grass, switchgrass, Spartina, rye grass, reed canary grass, miscanthus, residues from sugar processing, sugar cane bagasse, agricultural waste, rice straw, rice husks, barley straw, maize cob, straw of cereals, wheat straw, canola straw, oat straw, oat husks, maize cane, soybean flour, cornflour, forestry waste, recycled wood pulp fiber, paper sludge, sawdust, hardwood, resinous wood, agave and combinations thereof. In a preferred embodiment, the material containing lignocellulose is selected from a group comprising: cornflour, maize fiber, rice straw, pine wood, wood chips, poplar, bagasse, paper and waste from pulp treatment.

“Cellulose” means a linear homopolysaccharide derived from biomass (including organic materials of vegetable origin, including algae, cellulose of animal origin as well as cellulose of bacterial origin) and consisting of units (or rings) of glucose (D-anhydroglucopyranose—AGU for “Anhydroglucose unit”) joined together by β-(1-4) glycosidic bonds. The repeating unit is a glucose dimer, also called cellobiose.

The AGUs possess 3 hydroxyl functions: 2 secondary alcohols (on the carbons in positions 2 and 3 of the glucose ring) and a primary alcohol (on the carbon in position 6 of the glucose ring).

These polymers are joined together by intermolecular bonds of the hydrogen bond type, thus giving cellulose a fibrous structure. In particular, joining together of the chains formed from cellobiose dimers forms an elementary cellulose nanofibril (with diameter of about 5 nm). The association of elementary nanofibrils forms a nanofibril (with diameter generally varying from 50 to 500 nm; which includes 50, 60, 70, 80, 90, 100, 150, 200, 250, 300, 350, 400, 450 and 500 nm). Arrangement of several of these nanofibrils then forms what is generally called a cellulose fiber.

The term “cellulose fiber” denotes all of the forms of cellulose obtainable at the end of a process of defibrillation, or delamination of a cellulosic substrate; which includes the forms of cellulose having a size of the order of a nanometer, as well as the forms of cellulose having a larger size.

The term “nanocelluloses” denotes the various forms of cellulose having a size of the order of a nanometer. This term includes in particular, according to the invention, two families of nanocelluloses: cellulose nanocrystals and cellulose fibrils.

The terms “cellulose fibrils”, “(cellulose) nanofibrils”, “(cellulose) nanofibers”, “nanofibrillar cellulose”, “(cellulose) microfibrils”, “microfibrillar cellulose”, “microfibrillated cellulose”, “cellulose nanofibrils” are synonyms.

Each cellulose nanofibril contains crystalline parts stabilized by a sturdy network of hydrogen bonds between chains and within chains. These crystalline regions are separated by amorphous regions.

By removing the amorphous parts of the cellulose nanofibrils it is possible to obtain cellulose nanocrystals (CNCs).

The CNCs advantageously comprise at least 50% of crystalline part, more preferably at least 55% of crystalline part. They generally have a diameter in the range from 5 to 70 nm (preferably less than 15 nm) and a length from 40 nm to about 1 μm, preferably from 40 nm to 500 nm; which includes 50, 60, 70, 80, 90, 100, 150, 200, 250, 300, 350, 400, 450 and 500 nm.

The terms “cellulose nanocrystals”, “nanocrystalline cellulose”, “cellulose whiskers”, “microcrystals” or “cellulose nanocrystal” are synonyms. The term “cellulose nanocrystals” (CNCs) will generally be used hereinafter.

As used here, a “material containing polysaccharides” includes a substance or a composition comprising polysaccharides.

The term “polysaccharide” is used in its conventional sense and denotes polymeric carbohydrates made up of long chains of monosaccharides held together by glycosidic bonds. When they undergo hydrolysis, the polysaccharides release the soluble monosaccharides or oligosaccharides of which they are constituted.

The polysaccharides that are preferably considered are the polysaccharides derived from plants, and quite particularly cellulose, such as that found in lignocellulose.

This term thus includes any material (or substrate or biomass) comprising at least one (or a plurality of) polysaccharide (s) selected from: cellulose, hemicellulose and pectin; which in particular includes the polysaccharide substrates comprising at least 30 wt % of cellulose and hemicellulose.

The term “material containing lignocellulose” used here refers to a material mainly consisting of cellulose, hemicellulose and lignin. This term is a synonym of “lignocellulosic material”. Such a material is also referred to as “biomass”. According to this definition, a material containing lignocellulose is an example of a cellulosic substrate able to form cellulose fibers.

This term thus includes any lignocellulosic material (or substrate or biomass) containing at least 30 wt %, preferably at least 50 wt %, preferably at least 70 wt %, preferably at least 90 wt % of lignocellulose. In this respect, a lignocellulosic material may comprise other compounds such as polypeptides and sugars, which includes fermentable and/or nonfermentable sugars.

Thus, a material containing lignocellulose that is considered in particular according to the invention may be selected from pine, poplar, straw and miscanthus.

As used in the present application, an “enzyme oxidizing the polysaccharides” or a “polypeptide with polysaccharide oxidase activity” includes the polypeptides with the following properties:
said polypeptide produces hydrogen peroxide in the presence of oxygen and an electron-donating compound, such as ascorbates,
said polypeptide increases, in a dose-dependent manner, in the absence or in the presence of an electron-donating compound, the degradation of a material containing polysaccharides such as lignocellulose, caused by cellulases and/or hemicellulases such as, for example, the xylanases,
said polypeptide increases, in a dose-dependent manner, in the absence or in the presence of an electron-donating compound, the degradation of a material containing polysaccharides such as lignocellulose, caused by cellulases.

The term “electron-donating compound” is used here in its usual sense for a person skilled in the art. Thus, an electron-donating compound is a chemical entity capable of donating electrons to another compound. An electron-donating compound is a reducing agent owing to its capacity to donate electrons and is itself oxidized when it donates electrons to another chemical entity. An electron-donating compound as specified above for the properties of oxidation of polysaccharides, includes, nonexhaustively, ascorbates and cellobiose dehydrogenases (CDHs). In the absence of a reducing agent, such as ascorbate, the reducing agent may advantageously be supplied by the biomass (lignin), which can act as electron donor.

The term “LPMO”, or “Lytic Polysaccharide Monooxygenase”, denotes in its broadest sense, and unless stated otherwise, all of the families listed in the CAZy database (Carbohydrate Active enzymes, Lombard et al., 2014) and able to oxidize polysaccharides. This term therefore includes in particular all of the polypeptides with polysaccharide oxidase activity belonging to the families AA9, AA10, AA11, AA13, AA14 and AA15. This term also includes the LPMOs of type I (capable of oxidizing the glycosidic bonds on carbon Cl), of type II (capable of oxidizing the glycosidic bonds on carbon C4) and type III (capable of oxidizing the glycosidic bonds both at C1 and at C4).

The term “enzyme degrading the polysaccharides” or a “polypeptide with polysaccharide degradase activity” includes the polypeptides which, besides the polysaccharide oxidases, contribute to the degradation of the polysaccharide substrates such as lignocellulosic biomasses. Thus, polypeptides with polysaccharide degradase activity may be selected from the cellulases, hemicellulases, ligninases and carbohydrate oxidases.

The cellulases include the endoglucanases, cellobiohydrolases and beta-glucosidases. The hemicellulases include the xylanases, mannanases, xylosidases, mannosidases, arabinofuranosidases and esterases. Thus, the term “cellulase” may include the exo-glucanases, endo-glucanases, cellobiohydrolases, cellulose phosphorylases, pectinases, pectate lyases, polygalacturonase, pectin esterases, cellobiose dehydrogenases, mannanases, arabinofuranosidases, feruloyl esterases, arabinofuranosidases, fructofuranosidases, galactosidases, galactosidases, amylases, acetylxylan esterases, chitin deacetylases, chitinases, and glucosidases.

The ligninases include the peroxidases, copper radical oxidases (i.e. laccases). The carbohydrate oxidases include the lytic polysaccharide monooxygenases (LPMOs) and the GMC oxidoreductases (i.e. glucose dehydrogenases, cellobiose dehydrogenases).

The term “chemical treatment” refers to all the chemical pretreatments that allow the separation and/or release of cellulose, hemicellulose and/or lignin. Examples of suitable chemical pretreatments include, for example, dilute acids, lime, bases, organic solvents, ammonia, sulfur dioxide, carbon dioxide. Moreover, wet oxidation and hydrothermolysis at controlled pH are also regarded as chemical pretreatments. The methods of pretreatment using ammonia are described in particular in PCT applications WO 2006/110891, WO 2006/110899, WO 2006/110900, and WO 2006/110901.

Other examples of suitable pretreatment are described by Schell et al., 2003, Appl. Biochem and Biotechn. Vol. 105-108: 69-85, and Mosier et al., 2005, Bioresource Technology 96: 673-686, and U.S. Publication No. 2002/0164730.

The term “mechanical pretreatment” refers to all the mechanical (or physical) treatments that allow separation and/or release of cellulose, hemicellulose and/or lignin from a material containing lignocellulose. For example, the mechanical pretreatments include various types of grinding, irradiation, steam explosion and hydrothermolysis. Mechanical pretreatment includes fragmentation of a solid (comminution or mechanical size reduction). Fragmentation of a solid includes the techniques of dry grinding, wet grinding and vibrating ball grinding. The mechanical pretreatment may also include high pressures and/or high temperatures (steam explosion). In certain representations of the pretreatment step, said step may combine mechanical and chemical pretreatment.

As used in the present invention, the term “biological pretreatment” refers to all the biological treatments that allow separation and/or release of cellulose, hemicellulose and/or lignin from a material containing lignocellulose. The biological pretreatments may involve the application of microorganisms capable of solubilizing lignin (see, for example, Hsu, 1996, Pretreatment biomass, in Handbook on Bioethanol: Production and Utilization, Wyman, ed., Taylor & Francis, Washington, DC, 179-212; Ghosh and Singh, 1993, Physicochemical and biological treatments for enzymatic/microbial conversion of lignocellulosic biomass, Adv. Appl. Microbiol. 39: 295-333; McMillan, 1994, Pretreating lignocellulosic biomass: a review, in Enzymatic Conversion of Biomass for Fuels Production, Himmel, Baker, and Overend, eds., ACS Symposium Series 566, American Chemical Society, Washington, DC, chapter 15; Gong, Cao, Du, and Tsao, 1999, Ethanol production from renewable resources, in Advances in Biochemical Engineering/Biotechnology, Scheper, ed., Springer-Verlag Berlin Heidelberg, Germany, 65: 207-241; Olsson and Hahn-Hagerdal, 1996, Fermentation of lignocellulosic hydrolyzates for ethanol production, Enz. Microb. Tech. 18: 312-331; and Vallander and Eriksson, 1990, Production of ethanol from lignocellulosic material: State of the art, Adv. Biochem. Eng J Biotechnol. 42: 63-95).

Polypeptides and Compositions with Polysaccharide Oxidase Activity

According to a principal embodiment, the invention relates to an isolated polypeptide with polysaccharide oxidase activity, comprising a sequence having an amino acid identity of at least 20% with a reference polypeptide sequence SEQ ID No. 1 (derived from Podospora anserina) or SEQ ID No. 2 (derived from Aspergillus aculeatus), using a BLAST-P comparison method, said BLAST-P comparison method giving an e-value of 1e⁻¹⁸ or less.

In particular, the invention relates to an isolated polypeptide with polysaccharide oxidase activity, as defined above, characterized in that it comprises a sequence having an amino acid identity of at least 40% with a reference polypeptide sequence SEQ ID No. 1 or SEQ ID No. 2, said BLAST-P comparison method giving an e-value of 1e⁻¹⁸ or less.

For example, a polypeptide with polysaccharide oxidase activity according to the invention may be characterized in that it comprises a sequence having an amino acid identity of at least 45% with a reference polypeptide sequence SEQ ID No. 1 or SEQ ID No. 2, said BLAST-P comparison method giving an e-value of 1e⁻¹⁸ or less.

In particular, the invention relates to an isolated polypeptide with polysaccharide oxidase activity, as defined above, characterized in that it comprises a sequence having an amino acid identity of at least 60% with a reference polypeptide sequence SEQ ID No. 1 or SEQ ID No. 2, said BLAST-P comparison method giving an e-value of 1e⁻¹⁸ or less.

In particular, the invention relates to an isolated polypeptide with polysaccharide oxidase activity, as defined above, characterized in that it comprises a sequence having an amino acid identity of at least 80% with a reference polypeptide sequence SEQ ID No. 1 or SEQ ID No. 2, said BLAST-P comparison method giving an e-value of 1e⁻¹⁸ or less.

In particular, the invention relates to an isolated polypeptide with polysaccharide oxidase activity, as defined above, characterized in that it comprises a sequence having an amino acid identity of at least 90% with a reference polypeptide sequence SEQ ID No. 1 or SEQ ID No. 2, said BLAST-P comparison method giving an e-value of 1e⁻¹⁸ or less.

According to certain embodiments, the invention relates to one of said isolated polypeptides with polysaccharide oxidase activity, as defined above, characterized in that it comprises a reference polypeptide sequence having an amino acid identity of at least 20% with a sequence selected from SEQ ID No. 5 to SEQ ID No. 383, which includes SEQ ID No. 7 to 382 and SEQ ID No. 383.

According to certain embodiments, the invention relates to one of said isolated polypeptides with polysaccharide oxidase activity, as defined above, characterized in that it comprises a reference polypeptide sequence having an amino acid identity of at least 80% with a sequence selected from SEQ ID No. 5 to SEQ ID No. 383, which includes SEQ ID No. 7 to 382 and SEQ ID No. 383.

According to certain embodiments, the invention relates to one of said isolated polypeptides with polysaccharide oxidase activity, as defined above, characterized in that it comprises a reference polypeptide sequence having an amino acid identity of at least 90% with a sequence selected from SEQ ID No. 5 to SEQ ID No. 383, which includes SEQ ID No. 7 to 382 and SEQ ID No. 383.

For example, the invention relates to an isolated polypeptide with polysaccharide oxidase activity, as defined above, characterized in that it comprises a reference polypeptide sequence selected from SEQ ID No. 5 to SEQ ID No. 382 and SEQ ID No. 383.

According to a particular embodiment, the invention relates to an isolated polypeptide with polysaccharide oxidase activity, as defined above, characterized in that it comprises a sequence having an amino acid identity of at least 20% with a reference polypeptide sequence SEQ ID No. 5 or SEQ ID No. 6, said BLAST-P comparison method giving an e-value of 1e⁻¹⁸ or less.

According to a particular embodiment, the invention relates to an isolated polypeptide with polysaccharide oxidase activity, as defined above, characterized in that it comprises a sequence having an amino acid identity of at least 40% with a reference polypeptide sequence SEQ ID No. 5 or SEQ ID No. 6, said BLAST-P comparison method giving an e-value of 1e⁻¹⁸ or less.

For example, a polypeptide with polysaccharide oxidase activity according to the invention may be characterized in that it comprises a sequence having an amino acid identity of at least 45% with a reference polypeptide sequence SEQ ID No. 5 or SEQ ID No. 6, said BLAST-P comparison method giving an e-value of 1e⁻¹⁸ or less.

According to a particular embodiment, the invention relates to an isolated polypeptide with polysaccharide oxidase activity, as defined above, characterized in that it comprises a sequence having an amino acid identity of at least 60% with a reference polypeptide sequence SEQ ID No. 5 or SEQ ID No. 6, said BLAST-P comparison method giving an e-value of 1e⁻¹⁸ or less.

According to a particular embodiment, the invention relates to an isolated polypeptide with polysaccharide oxidase activity, as defined above, characterized in that it comprises a sequence having an amino acid identity of at least 80% with a reference polypeptide sequence SEQ ID No. 5 or SEQ ID No. 6, said BLAST-P comparison method giving an e-value of 1e⁻¹⁸ or less.

According to a particular embodiment, the invention relates to an isolated polypeptide with polysaccharide oxidase activity, as defined above, characterized in that it comprises a sequence having an amino acid identity of at least 90% with a reference polypeptide sequence SEQ ID No. 5 or SEQ ID No. 6, said BLAST-P comparison method giving an e-value of 1e⁻¹⁸ or less.

According to an alternative embodiment, the invention relates to a composition with polysaccharide oxidase activity, characterized in that it comprises a polypeptide with polysaccharide oxidase activity as defined above.

In particular, the invention relates to a composition with polysaccharide oxidase activity as defined above, characterized in that it further comprises at least one polypeptide with polysaccharide degradase activity selected from: a cellulase, a hemicellulase, a ligninase and a carbohydrate oxidase.

Nonexhaustively, said composition with polysaccharide oxidase activity may be characterized in that it is obtainable from one or more organisms selected from: Agaricus bisporus, Alternaria alternata, Amanita thiersii, Armillaria ostoyae, Armillaria solidipes, Ascochyta rabiei, Aspergillus arachidicola, Aspergillus bombycis, Aspergillus brasiliensis, Aspergillus campestris, Aspergillus candidus, Aspergillus carbonarius, Aspergillus clavatus, Aspergillus cristatus, Aspergillus fischeri, Aspergillus flavus, Aspergillus fumigatus, Aspergillus glaucus, Aspergillus kawachii, Aspergillus lentulus, Aspergillus luchuensis, Aspergillus nidulans, Aspergillus niger, Aspergillus nomius, Aspergillus novofumigatus, Aspergillus ochraceoroseus, Aspergillus oryzae, Aspergillus parasiticus, Aspergillus ruber, Aspergillus steynii, Aspergillus sydowii, Aspergillus taichungensis, Aspergillus terreus, Aspergillus thermomutatus, Aspergillus tubingensis, Aspergillus turcosus, Aspergillus udagawae, Aspergillus versicolor, Aspergillus wentii, Aureobasidium melanogenum, Aureobasidium namibiae, Aureobasidium pullulans, Aureobasidium subglaciale, Auricularia subglabra, Bipolaris maydis, Bipolaris oryzae, Bipolaris sorokiniana, Bipolaris victoriae, Bipolaris zeicola, Botryobasidium botryosum, Botrytis cinerea, Ceratocystis fimbriata, Ceratocystis platani, Cercospora berteroae, Cercospora beticola, Cercospora zeina, Chaetomium globosum, Chaetomium globosum, Clohesyomyces aquaticus, Colletotrichum chlorophyti, Colletotrichum fioriniae, Colletotrichum gloeosporioides, Colletotrichum graminicola, Colletotrichum higginsianum, Colletotrichum incanum, Colletotrichum nymphaeae, Colletotrichum orbiculare, Colletotrichum orchidophilum, Colletotrichum salicis, Colletotrichum simmondsii, Colletotrichum sublineola, Colletotrichum tofieldiae, Coniella lustricola, Coniochaeta ligniaria, Corynespora cassiicola, Cylindrobasidium torrendii, Daldinia sp., Diaporthe ampelina, Diaporthe helianthi, Diplocarpon rosae, Diplodia corticola, Diplodia seriata, Dothistroma septosporum, Elsinoe, Elsinoe australis, Emergomyces pasteuriana, Epicoccum nigrum, Eutypa lata, Exidia glandulosa, Fungi, Fusarium avenaceum, Fusarium culmorum, Fusarium fujikuroi, Fusarium graminearum, Fusarium langsethiae, Fusarium mangiferae, Fusarium nygamai, Fusarium oxysporum, Fusarium poae, Fusarium proliferatum, Fusarium pseudograminearum, Fusarium venenatum, Fusarium verticillioides, Gaeumannomyces tritici, Glarea lozoyensis, Glonium stellatum, Grosmannia clavigera, Helicocarpus griseus, Heterobasidion irregulare, Hirsutella minnesotensis, Histoplasma capsulatum, Hortaea werneckii, Hydnomerulius pinastri, Hypoxylon sp., Jaapia argillacea, Leptosphaeria maculans, Leucoagaricus, Lomentospora prolificans, Magnaporthe oryzae, Magnaporthiopsis poae, Marssonina brunnea, Marssonina coronariae, Meliniomyces bicolor, Meliniomyces variabilis, Microdochium bolleyi, Moniliophthora roreri, Mycosphaerella eumusae, Neofusicoccum parvum, Neonectria ditissima, Ophiocordyceps australis, Ophiocordyceps camponotirufipedis, Ophiocordyceps unilateralis, Panaeolus cyanescens, Paraphaeosphaeria sporulosa, Parastagonospora nodorum, Penicilliopsis zonata, Penicillium arizonense, Penicillium brasilianum, Penicillium camemberti, Penicillium coprophilum, Penicillium digitatum, Penicillium expansum, Penicillium flavigenum, Penicillium freii, Penicillium griseofulvum, Penicillium italicum, Penicillium nordicum, Penicillium oxalicum, Penicillium polonicum, Penicillium roqueforti, Penicillium rubens, Penicillium solitum, Penicillium subrubescens, Penicillium vulpinum, Peniophora, Pestalotiopsis fici, Phellinus noxius, Phialocephala, scopiformis, Phialocephala, subalpina, Pleurotus ostreatus, Plicaturopsis crispa, Pseudogymnoascus verrucosus, Pseudomassariella vexata, Punctularia strigosozonata, Pyrenochaeta, Pyrenophora teres, Pyrenophora triticirepentis, Rhizoctonia solani, Rhynchosporium commune, Rosellinia necatrix, Rutstroaemia, Scedosporium apiospermum, Schizophyllum commune, Sclerotinia borealis, Sclerotinia sclerotiorum, Serpula lacrymans, Setosphaeria turcica, Sordaria macrospora, Sphaerulina musiva, Sporothrix brasiliensis, Sporothrix insectorum, Sporothrix schenckii, Stachybotrys chartarum, Stachybotrys chlorohalonata, Stagonospora, Stemphylium lycopersici, Thermothelomyces thermophila, Thielavia terrestris, Thielaviopsis punctulata, Valsa mali, Verruconis gallopava, Verticillium alfalfae, Verticillium dahliae, Verticillium longisporum.

In particular, said composition with polysaccharide oxidase activity may be characterized in that it is obtainable from one or more organisms of the type bacterium, fungus (for example filamentous fungi) or yeast, selected from the genera: Escherichia, Lactococcus, Bacillus, Streptomyces, Pseudomonas, Phanerochaete, Achlya, Acremonium, Aspergillus, Cephalosporium, Chrysosporium, Cochliobolus, Endothia, Fusarium, Gliocladium, Humicola, Hypocrea, Myceliophthora, Mucor, Neurospora, Penicillium, Pyricularia, Thielavia, Tolypocladium, Trichoderma, Podospora, Pycnoporus, Fusarium, Thermonospora; quite particularly Aspergillus or Trichoderma or Podospora; preferably Aspergillus or Podospora.

In particular, said composition with polysaccharide oxidase activity may be characterized in that it is obtainable from one or more organisms of the fungus or yeast type, selected from the genera: Achlya, Acremonium, Aspergillus, Cephalosporium, Chrysosporium, Cochliobolus, Endothia, Fusarium, Gliocladium, Humicola, Hypocrea, Myceliophthora, Mucor, Neurospora, Penicillium, Pyricularia, Thielavia, Tolypocladium, Trichoderma, Podospora, Pycnoporus, Fusarium, Thermonospora; quite particularly Aspergillus or Trichoderma or Podospora; preferably Aspergillus or Podospora.

According to one embodiment, said composition with polysaccharide oxidase activity may be characterized in that it is obtainable from Aspergillus japonicus.

A polypeptide with polysaccharide oxidase activity according to the invention may also be obtained recombinantly.

Thus, a composition with polysaccharide oxidase activity according to the invention may be characterized in that said polypeptide with polysaccharide oxidase activity is obtained recombinantly.

According to certain embodiments, a composition with polysaccharide oxidase activity according to the invention comprises only one polypeptide (or “enzyme”) with polysaccharide oxidase activity according to the invention, or a polypeptide having a sequence having an amino acid identity of at least 20% with a reference polypeptide sequence SEQ ID No. 1 (derived from Podospora anserina) or SEQ ID No. 2 (derived from Aspergillus aculeatus), using a BLAST-P comparison method, said BLAST-P comparison method giving an e-value of 1e⁻¹⁸ or less.

According to certain embodiments, a composition with polysaccharide oxidase activity according to the invention comprises a plurality of polypeptides with polysaccharide oxidase activity according to the invention, or more than one polypeptide having a sequence having an amino acid identity of at least 20% with a reference polypeptide sequence SEQ ID No. 1 (derived from Podospora anserina) or SEQ ID No. 2 (derived from Aspergillus aculeatus), using a BLAST-P comparison method, said BLAST-P comparison method giving an e-value of 1e⁻¹⁸ or less.

According to certain of these embodiments, a composition with polysaccharide oxidase activity according to the invention comprises at least 2, 3, 4, 5, 6, 7, 8, 9 or 10 polypeptides with polysaccharide oxidase activity according to the invention.

According to certain embodiments, a composition with polysaccharide oxidase activity according to the invention further comprises at least one other polypeptide with polysaccharide oxidase activity; in particular selected from the lytic polysaccharide monooxygenases, which includes the polypeptides belonging to the LPMO groups AA9, AA10, AA11, AA13 and AA14.

According to certain embodiments, a composition with polysaccharide oxidase activity according to the invention may be used as a composition for degrading a polysaccharide substrate, such as lignocellulosic biomasses.

According to certain other embodiments, a composition with polysaccharide oxidase activity according to the invention may be used as an auxiliary composition in combination with one or more polypeptides with polysaccharide degradase activity.

Thus, according to an alternative embodiment, a polypeptide with polysaccharide oxidase activity according to the invention may be used in the form of a kit.

Advantageously, a kit of this kind may comprise a polypeptide with polysaccharide oxidase activity or a composition with polysaccharide oxidase activity according to the invention; and in addition a polypeptide with polysaccharide degradase activity or a composition comprising said polypeptide with polysaccharide degradase activity.

Thus, the invention also relates to a kit comprising at least:
a first part comprising a polypeptide with polysaccharide oxidase activity according to the invention, or a composition comprising said polypeptide with polysaccharide oxidase activity;
a second part comprising a polypeptide with polysaccharide degradase activity selected from a cellulase, a hemicellulase, a ligninase and a carbohydrate oxidase, or a composition comprising said polypeptide with polysaccharide degradase activity.

Such kits may correspond in particular to kits for:
obtaining a sugar starting from a polysaccharide substrate;
preparing a fermentation product from a polysaccharide substrate;
producing alcohol starting from a lignocellulosic substrate;
preparing a cellulosic substrate for producing cellulose fibers;
defibrillating a cellulosic substrate;
producing cellulose fibers.

According to another embodiment, the invention relates to a host able to express recombinantly a polypeptide with polysaccharide oxidase activity according to the invention. Said host may in particular be a prokaryotic or eukaryotic cell. According to a particular embodiment, said host is a yeast or a bacterium or a fungus, for example a filamentous fungus.

According to the invention, a “host” may in particular be a yeast selected from a group comprising: Saccharomyces, Kluyveromyces, Candida, Pichia, Schizosaccharomyces, Hansenula, Kloeckera, Schwanniomyces, and Yarrowia.

In particular, the yeasts may be selected from: S. cerevisiae, S. bulderi, S. barnetti, S. exiguus, S. uvarum, S. diastaticus, K. lactis, K. marxianus, or K. fragilis.

According to certain embodiments, the yeast is selected from: Saccharomyces cerevisiae, Schizzosaccharomyces pombe, Issatchenkia orientalis, Candida albicans, Candida mexicana, Pichia pastoris, Pichia mississippiensis, Pichia mexicana, Pichia stipitis, Pichia farinosa, Clavispora opuntiae, Clavispora lusitaniae, Yarrowia lipolytica, Hansenula polymorpha, Phaffia rhodozyma, Candida utilis, Arxula adeninivorans, Debaryomyces hansenii, Debaryomyces polymorphus, Schizosaccharomyces pombe, Hansenula polymorpha, and Schwanniomyces occidentalis.

According to other embodiments, a “host” may in particular be a bacterium selected from a group comprising: Escherichia, Lactococcus, Bacillus, Streptomyces, Pseudomonas.

According to other embodiments, a “host” may in particular be a fungus selected from a group comprising: Aspergillus, Trichoderma, Podospora, Myceliophthora, Chrysosporium, Neurospora, Fusarium, Phanerochaete, Penicillium.

According to a preferred embodiment, said host is a yeast.

The invention also relates to an isolated nucleic acid coding for a polypeptide with polysaccharide oxidase activity according to the invention.

Thus, a nucleic acid allowing expression of a polypeptide with polysaccharide oxidase activity according to the invention may be introduced into the genome of a host (i.e. yeast) of interest, or else introduced as a non-integrated vector according to techniques of genetic engineering known in this field.

The invention also relates to a method for producing a polypeptide with polysaccharide oxidase activity; comprising a step of culturing a host capable of expressing a polypeptide with polysaccharide oxidase activity according to the invention.

Thus, the invention relates to vectors including a nucleic acid coding for a polypeptide with polysaccharide oxidase activity according to the invention. In particular, said vectors (i.e. plasmids or YAC) are expression vectors capable of directing the expression of a given gene, and associated with an operon.

The invention therefore also relates to a method for producing a polypeptide with polysaccharide oxidase activity; consisting of:
(a) culturing a host capable of producing a polypeptide with polysaccharide oxidase activity, characterized in that it comprises a sequence having an amino acid identity of at least 20% with a reference polypeptide sequence SEQ ID No. 1 (derived from Podospora anserina) or SEQ ID No. 2 (derived from Aspergillus aculeatus), using a BLAST-P comparison method, said BLAST-P comparison method giving an e-value of 1e⁻¹⁸ or less; and
(b) recovering said polypeptide from said host.
   Methods of Obtaining Sugars Starting from Polysaccharide Substrates

The polypeptides with polysaccharide oxidase activity according to the invention may be used both in methods for obtaining a sugar starting from a polysaccharide substrate, methods of fermentation and of production of alcohol starting from a lignocellulosic substrate.

Thus, methods for producing ethanol starting from polysaccharide substrates, such as lignocellulosic biomasses, are described in Margeot et al. (“New improvements for lignocellulosic ethanol”; Current Opinion in Biotechnology; 20:372-380, 2009). Thus, a complete method for ethanol production starting from lignocellulosic biomass generally comprises four main steps:
1. pretreatment of the polysaccharide substrate (i.e. lignocellulosic biomass); for breaking the structure of the lignocellulosic wall, by liquefying certain components and by reducing the crystallinity of the cellulose.

There are physical (mechanical or hydrothermal) pretreatments for reducing the particle size and dissolving a portion of the hemicelluloses, chemical pretreatments that use acids, bases or oxidizing agents for solubilizing the hemicelluloses and removing the lignin, and biological pretreatments that use fungal species capable of degrading lignin. Certain pretreatments combine physical and chemical methods, for example such as steam explosion in the presence of dilute acid or cold explosion with ammonia.
2. enzymatic hydrolysis of said substrate, consisting of depolymerizing the cellulose and the remaining hemicelluloses into monomers, using specific enzymes.

These enzymes are secreted by a variety of cellulolytic, aerobic or anaerobic, mesophilic or thermophilic organisms, belonging in particular to the genera Clostridium, Thermomonospora, Cellulomonas, Bacteroides or Streptomyces for the bacteria, and Phanerochaete, Trichoderma, Aspergillus or Penicillium for the filamentous fungi.
3. fermentation of said substrate to obtain said ethanol starting from sugars (i.e. pentoses and hexoses) resulting from the hydrolysis step.

This fermentation step is in particular carried out with microorganisms such as the yeast Saccharomyces cerevisiae, which is the microorganism most used in industry owing to its high yields and low sensitivity to inhibitors. However, it is not capable naturally of utilizing the pentoses produced during enzymatic hydrolysis. Thus, in order to improve yields, modified strains capable of fermenting sugars such as xylose or arabinose have been developed.
4. purification of said ethanol, generally with a view to using it as motor fuel, which must meet a certain number of requirements. This purification generally consists of distillation, rectification and dehydration.

According to one embodiment, these steps may be carried out separately, or else simultaneously. For example, these various steps may be carried out separately, as is the case in a method designated SHF (“Separate Hydrolysis and Fermentation”) in which the enzymes and the yeasts may each be used in their optimal conditions. It is also possible to perform these steps simultaneously, by a method called SSCF (“Simultaneous Saccharification and Co-Fermentation”). Finally, the concept of “Consolidated Bioprocessing” (CBP) proposes carrying out the production of cellulases, enzymatic hydrolysis and fermentation using a single microorganism.

To make lignocellulosic bioethanol cost-effective, it is generally necessary to optimize each of the steps in its production. It is in particular essential to select an appropriate pretreatment suitable for the type of biomass being treated, in order to maximize its effect, while limiting the formation of inhibitors and reducing costs and energy consumption. Enzymatic hydrolysis is often regarded as the most critical step in terms of yields and cost.

The polypeptides with polysaccharide oxidase activity according to the invention are particularly advantageous in the context of a step of hydrolysis of polysaccharide substrates.

Thus, according to one embodiment, the invention relates to a method of obtaining a sugar starting from a polysaccharide substrate, comprising at least one step of contacting said substrate with a polypeptide with polysaccharide oxidase activity, comprising a sequence having an amino acid identity of at least 20% with a reference polypeptide sequence SEQ ID No. 1 (derived from Podospora anserina) or SEQ ID No. 2 (derived from Aspergillus aculeatus), using a BLAST-P comparison method, said BLAST-P comparison method giving an e-value of 1e⁻¹⁸ or less, or a composition with polysaccharide oxidase activity comprising said polypeptide.

In particular, the invention relates to a method of obtaining a sugar starting from a polysaccharide substrate, comprising at least one step of contacting said substrate with a polypeptide with polysaccharide oxidase activity, comprising a sequence having an amino acid identity of at least 20% with a reference polypeptide sequence SEQ ID No. 5 (derived from Podospora anserina) or SEQ ID No. 6 (derived from Aspergillus aculeatus), using a BLAST-P comparison method, said BLAST-P comparison method giving an e-value of 1e⁻¹⁸ or less, or a composition with polysaccharide oxidase activity comprising said polypeptide.

Thus, the invention relates to a method of obtaining a sugar starting from a polysaccharide substrate, comprising the steps of:
a) providing a polysaccharide substrate;
b) contacting said polysaccharide substrate with a polypeptide with polysaccharide oxidase activity, comprising a sequence having an amino acid identity of at least 20% with a reference polypeptide sequence SEQ ID No. 1 (derived from Podospora anserina) or SEQ ID No. 2 (derived from Aspergillus aculeatus), using a BLAST-P comparison method, said BLAST-P comparison method giving an e-value of 1e⁻¹⁸ or less, or a composition with polysaccharide oxidase activity comprising said polypeptide;
c) collecting said sugar obtained at the end of step b).

In particular, the invention relates to a method of obtaining a sugar starting from a polysaccharide substrate, comprising the steps of:
a) providing a polysaccharide substrate;
b) contacting said polysaccharide substrate with a polypeptide with polysaccharide oxidase activity, comprising a sequence having an amino acid identity of at least 20% with a reference polypeptide sequence SEQ ID No. 5 (derived from Podospora anserina) or SEQ ID No. 6 (derived from Aspergillus aculeatus), using a BLAST-P comparison method, said BLAST-P comparison method giving an e-value of 1e⁻¹⁸ or less, or a composition with polysaccharide oxidase activity comprising said polypeptide;
c) collecting said sugar obtained at the end of step b).

In particular, the invention relates to a method of obtaining a sugar as defined above, characterized in that said substrate is a lignocellulosic substrate, and quite particularly a lignocellulosic biomass.

According to another embodiment, the invention relates to a method for preparing a fermentation product starting from a polysaccharide substrate, characterized in that it comprises obtaining a sugar by a method as defined above, and fermentation of said sugar so as to obtain a fermentation product.

Thus, the invention relates method for preparing a fermentation product starting from a polysaccharide substrate, characterized in that it comprises the following steps:
a) contacting said substrate with a polypeptide with polysaccharide oxidase activity, comprising a sequence having an amino acid identity of at least 20% with a reference polypeptide sequence SEQ ID No. 1 (derived from Podospora anserina) or SEQ ID No. 2 (derived from Aspergillus aculeatus), using a BLAST-P comparison method, said BLAST-P comparison method giving an e-value of 1e⁻¹⁸ or less, or a composition with polysaccharide oxidase activity comprising said polypeptide, so as to obtain a sugar;
b) fermenting said sugar so as to obtain a fermentation product.

In particular, the invention relates to a method for preparing a fermentation product starting from a polysaccharide substrate, characterized in that it comprises the following steps:
a) contacting said substrate with a polypeptide with polysaccharide oxidase activity, comprising a sequence having an amino acid identity of at least 20% with a reference polypeptide sequence SEQ ID No. 5 (derived from Podospora anserina) or SEQ ID No. 6 (derived from Aspergillus aculeatus), using a BLAST-P comparison method, said BLAST-P comparison method giving an e-value of 1e⁻¹⁸ or less, or a composition with polysaccharide oxidase activity comprising said polypeptide, so as to obtain a sugar;
b) fermenting said sugar so as to obtain a fermentation product.

According to another embodiment, the invention relates to a method of producing alcohol starting from a lignocellulosic substrate, characterized in that it comprises obtaining a sugar by a method as defined above, and alcoholic fermentation of said sugar by an alcohol-producing microorganism.

The fermentation product is preferably butanol, ethanol, isopropanol or a mixture thereof.

Thus, in the context of ethanolic fermentation, the product of alcoholic fermentation considered is ethanol.

Thus, preferably, a method of fermentation and/or production of alcohol according to the invention is a method of production of butanol, ethanol, isopropanol or a mixture thereof.

Thus, the invention relates to a method of producing alcohol starting from a lignocellulosic substrate, characterized in that it comprises the following steps:
a) contacting said substrate with a polypeptide with polysaccharide oxidase activity, comprising a sequence having an amino acid identity of at least 20% with a reference polypeptide sequence SEQ ID No. 1 (derived from Podospora anserina) or SEQ ID No. 2 (derived from Aspergillus aculeatus), using a BLAST-P comparison method, said BLAST-P comparison method giving an e-value of 1e⁻¹⁸ or less, or a composition with polysaccharide oxidase activity comprising said polypeptide, so as to obtain a sugar;
b) fermenting said sugar by an alcohol-producing microorganism so as to produce said alcohol.

In particular, the invention relates to a method of producing alcohol starting from a lignocellulosic substrate, characterized in that it comprises the following steps:
a) contacting said substrate with a polypeptide with polysaccharide oxidase activity, comprising a sequence having an amino acid identity of at least 20% with a reference polypeptide sequence SEQ ID No. 5 (derived from Podospora anserina) or SEQ ID No. 6 (derived from Aspergillus aculeatus), using a BLAST-P comparison method, said BLAST-P comparison method giving an e-value of 1e⁻¹⁸ or less, or a composition with polysaccharide oxidase activity comprising said polypeptide, so as to obtain a sugar;
b) fermenting said sugar by an alcohol-producing microorganism so as to produce said alcohol.

### Cellulose Fibers, Methods for Preparing Substrates and for Defibrillation

Various forms of cellulose were identified with a size of the order of a nanometer, designated with the general name “nanocelluloses”. The properties, especially mechanical, of cellulose fibers, including these nanocelluloses, their capacity to form films and their viscosity, also make them of major interest in many fields of industry. Several strategies for pretreating cellulose fibers have thus been developed in order to reduce the energy consumption required for their mechanical delamination.

The methods defined hereunder relate to obtaining cellulose fibers starting from a cellulosic substrate. They involve using a polypeptide with polysaccharide oxidase activity according to the invention.

According to one embodiment, the invention relates to a method for preparing a cellulosic substrate for producing cellulose fibers, said method comprising at least the following steps consisting of:
a) providing a cellulosic substrate able to form cellulose fibers;
b) in the presence of an electron donor, contacting said substrate with at least one polypeptide with polysaccharide oxidase activity, in conditions suitable for ensuring oxidative cleavage of said cellulose fibers; characterized in said that polypeptide with polysaccharide oxidase activity has an amino acid identity of at least 20% with a reference polypeptide sequence SEQ ID No. 1 or SEQ ID No. 2, using a BLAST-P comparison method, said BLAST-P comparison method giving an e-value of 1e⁻¹⁸ or less.

According to a particular embodiment, the invention relates to a method for preparing a cellulosic substrate for producing cellulose fibers, said method comprising at least the following steps consisting of:
a) providing a cellulosic substrate able to form cellulose fibers;
b) in the presence of an electron donor, contacting said substrate with at least one polypeptide with polysaccharide oxidase activity, in conditions suitable for ensuring oxidative cleavage of said cellulose fibers; characterized in that said polypeptide with polysaccharide oxidase activity has an amino acid identity of at least 20% with a reference polypeptide sequence SEQ ID No. 5 or SEQ ID No. 6, using a BLAST-P comparison method, said BLAST-P comparison method giving an e-value of 1e⁻¹⁸ or less.

According to one embodiment, the invention relates to a method of defibrillating a cellulosic substrate, said method comprising at least the following steps consisting of:
a) providing a cellulosic substrate able to form cellulose fibers, which had been brought into contact beforehand with an electron donor and a polypeptide with polysaccharide oxidase activity, in conditions suitable for ensuring oxidative cleavage of said cellulose fibers in the presence of an electron donor; and
b) treating said cellulosic substrate mechanically, in order to defibrillate said substrate, characterized in that said polypeptide with polysaccharide oxidase activity has an amino acid identity of at least 20% with a reference polypeptide sequence SEQ ID No. 1 or SEQ ID No. 2, using a BLAST-P comparison method, said BLAST-P comparison method giving an e-value of 1e⁻¹⁸ or less.

According to a particular embodiment, the invention relates to a method of defibrillating a cellulosic substrate, said method comprising at least the following steps consisting of:
a) providing a cellulosic substrate able to form cellulose fibers, which had been brought into contact beforehand with an electron donor and a polypeptide with polysaccharide oxidase activity, in conditions suitable for ensuring oxidative cleavage of said cellulose fibers in the presence of an electron donor; and
b) treating said cellulosic substrate mechanically, in order to defibrillate said substrate, characterized in that said polypeptide with polysaccharide oxidase activity has an amino acid identity of at least 20% with a reference polypeptide sequence SEQ ID No. 5 or SEQ ID No. 6, using a BLAST-P comparison method, said BLAST-P comparison method giving an e-value of 1e⁻¹⁸ or less.

According to one embodiment, the invention relates to a method of producing cellulose fibers, said method comprising at least the following steps consisting of:
a) providing a cellulosic substrate able to form cellulose fibers;
b) in the presence of an electron donor, contacting said cellulosic substrate with at least one polypeptide with polysaccharide oxidase activity in conditions suitable for ensuring oxidative cleavage of said cellulose fibers;
c) treating said cellulosic substrate mechanically, in order to produce said cellulose fibers starting from said cellulosic substrate, characterized in said that polypeptide with polysaccharide oxidase activity has an amino acid identity of at least 20% with a reference polypeptide sequence SEQ ID No. 1 or SEQ ID No. 2, using a BLAST-P comparison method, said BLAST-P comparison method giving an e-value of 1e⁻¹⁸ or less.

According to a particular embodiment, the invention relates to a method of producing cellulose fibers, said method comprising at least the following steps consisting of:
a) providing a cellulosic substrate able to form cellulose fibers;
b) in the presence of an electron donor, contacting said cellulosic substrate with at least one polypeptide with polysaccharide oxidase activity in conditions suitable for ensuring oxidative cleavage of said cellulose fibers;
c) treating said cellulosic substrate mechanically, in order to produce said cellulose fibers starting from said cellulosic substrate, characterized in that said polypeptide with polysaccharide oxidase activity has an amino acid identity of at least 20% with a reference polypeptide sequence SEQ ID No. 5 or SEQ ID No. 6, using a BLAST-P comparison method, said BLAST-P comparison method giving an e-value of 1e⁻¹⁸ or less.

Said polypeptide with polysaccharide oxidase activity according to the invention is mixed with the cellulosic substrate, so as to allow contacting between said polypeptide and the cellulose fibers.

The step of enzymatic treatment is preferably carried out with gentle stirring, so as to ensure good dispersion of the enzymes within the fibers. This step of enzymatic treatment is for example carried out for a time ranging from 24 h to 72 h (preferably 48 h).

Preferably, the step of enzymatic treatment is in carried out at a temperature from 30 to 50° C., particular from 30 to 45° C.

The pH of the reaction conditions of the enzyme in contact with the cellulosic substrate is generally between 3 and 7, which includes between 4 and 7, and in particular between 4 and 6.

According to the invention, said polypeptide may be added to the cellulosic substrate at an enzyme/cellulose ratio from 1:1000 to 1:50, in particular from 1:500 to 1:50 or from 1:100 to 1:50 or else from 1:1000 to 1:500, from 1:500 to 1:100.

Preferably, said polypeptide is used at a concentration from 0.001 to 10 g/L, in particular from 0.1 to 5 g/L, and more preferably from 0.5 to 5 g/L.

According to a particular embodiment, the cellulosic substrate is submitted to at least two (or even only two) successive steps of enzymatic treatment (in series, advantageously separated by a rinsing step).

The polypeptide or polypeptides used in each of these steps of enzymatic treatment are identical or different; the conditions (in particular the enzyme/substrate ratio) are identical or different between these successive steps.

Non-exclusively, tests of cleavage of cellulose by a polypeptide according to the invention may be carried out according to the following protocol:

For example, a cleavage test may be carried out in a volume of 300 μl of liquid containing 4.4 μM of LPMO enzyme and 1 mM of ascorbate and 0.1% (weight/volume) of cellulose powder swollen with phosphoric acid (PASC, phosphoric acid-swollen cellulose—prepared as described in Wood TM, Methods Enzym 1988, 160: 19-25) in 50 mM of a sodium acetate buffer at pH 4.8 or 5 μM of cello-oligosaccharides (Megazyme, Wicklow, Ireland) in 10 mM of sodium acetate buffer at pH 4.8.

The enzymatic reaction may be carried out in a 2-ml tube incubated in a thermomixer (Eppendorf, Montesson, France) at 50° C. and 580 rpm (revolutions per minute).

The fibers are brought into contact with the enzymes (at a concentration between 1 and 5 g/L and at enzyme/cellulose ratios of 1:50, 1:100, 1:500 and 1:1000) and the ascorbate (2 mM) and then stirred gently for 48 hours at 40° C.

After incubation for 16 h, the sample is heated to 100° C. for 10 minutes to stop the enzymatic reaction, and then centrifuged at 16 000 revolutions per minute (rpm) for 15 minutes at 4° C. in order to separate the solution fraction from the remaining insoluble fraction.

The treated fibers are then submitted to mechanical action with a homogenizing-dispersing machine (Ultra-Turrax, power 500 W, maximum speed for 3 minutes), followed by ultrasonic treatment for 3 minutes.

According to one embodiment, said methods are characterized in that the cellulose fibers obtained as a result of the procedure are cellulose nanofibrils.

According to one embodiment, said methods are characterized in that the electron donor is selected from ascorbate, gallate, catechol, reduced glutathione, lignin fragments and fungal carbohydrate dehydrogenases; and preferably ascorbate.

According to one embodiment, said methods are characterized in that the cellulosic substrate is obtained from wood, a cellulose-rich fibrous plant, beet, citrus fruits, annual straw plants, marine animals, algae, fungi or bacteria.

According to one embodiment, said methods are characterized in that the cellulosic substrate is selected from the chemical papermaking pulps, preferably chemical wood pulps, more preferably at least one of the following papermaking pulps: bleached pulps, semibleached pulps, unbleached pulps, bisulfite pulps, sulfate pulps, sodium hydroxide pulps, kraft pulps.

According to one embodiment, said methods are characterized in that the cellulosic substrate is a papermaking pulp derived from wood, annual plants or fiber plants.

According to one embodiment, said methods are characterized in that said at least one step of mechanical treatment comprises at least one of the following mechanical treatments:
a homogenization treatment,
a microfluidization treatment,
an abrasion treatment, and/or
a cryogrinding treatment.

According to one embodiment, said methods are characterized in that, following said step of mechanical treatment, said method comprises a step of posttreatment selected from: acid treatment, enzymatic treatment, oxidation, acetylation, silylation, or else derivatization of chemical groups carried by said cellulose fibers.

According to another aspect, the invention relates to cellulose fibers resulting from a defibrillation process and/or a method of producing cellulose fibers as defined above.

Said cellulose fibers may be characterized in that said cellulose fibers, preferably nanocellulose fibers, comprise glucose rings, at least one carbon atom of which is oxidized in position (s) C₁ and/or C₄, or also C₆.

According to a preferred embodiment, the cellulose fibers are cellulose nanofibrils.

### Step (s) of Mechanical Treatment(s)

The cellulosic substrate brought into contact with said enzyme is then submitted to at least one step of mechanical treatment that is intended to delaminate the cellulose fibers to obtain nanocelluloses.

Delamination (also called “fibrillation” or “defibrillation”) consists of separating, by a mechanical effect, the cellulose fibers within the cellulosic substrate, in particular for producing nanocelluloses.

As demonstrated, the oxidative cleavage of cellulose fibers, catalyzed by said at least one LPMO, facilitates delamination of these cellulose fibers during the step of mechanical treatment.

This step of mechanical delamination of the cellulose fibers may thus be carried out in conditions that are less harsh and therefore less costly in terms of energy.

The mechanical treatments with the aim of delaminating cellulose fibers are known by a person skilled in the art and may be employed in the method (s) of the invention.

In general, we may mention the low-level mechanical treatments with a homogenizing-dispersing machine (for example of the Ultra-Turrax type) and/or ultrasonic treatments.

We may for example also refer to the document of Lavoine N et al. (Carbohydrate Polymers, 2012, (92): 735-64), which describes in particular (pages 740 to 744) mechanical treatments for preparing microfibrillar cellulose (for example cellulose nanofibrils).

Typically, a mechanical treatment may be selected from mechanical treatments of homogenization, microfluidization, abrasion, or cryogrinding.

The homogenization treatment involves passing the pretreated cellulosic substrate, typically a cellulose pulp or a liquid suspension of cellulose, through a narrow space under high pressure (as described for example in patent U.S. Pat. No. 4,486,743).

This homogenization treatment is preferably carried out by means of a homogenizer of the Gaulin type. In such a device, the pretreated cellulosic substrate, typically in the form of a suspension of cellulose, is pumped at high pressure and distributed through a small-orifice automatic valve. A rapid succession of openings and closures of the valve subjects the fibers to a large pressure drop (generally of at least 20 MPa) and a high-speed shearing action followed by an impact of deceleration at high speed. Passage of the substrate through the orifice is repeated (generally from 8 to 10 times) until the cellulose suspension becomes stable. Cooling water is generally used in order to maintain a temperature of the product in a range from 70 to 80° C. during the homogenization treatment.

This homogenization treatment may also be carried out using a device of the microfluidizer type (see for example Sisqueira et al. Polymer 2010 2 (4): 728-65). In such a device, the cellulose suspension passes through a fine chamber typically of “z” shape (in which the size of the channel is generally between 200 and 400 μm) under high pressure (about 2070 bar). The high shear rate that is applied (generally greater than 10⁷·s⁻¹) makes it possible to obtain very fine nanofibrils. A variable number of passes (for example from 2 to 30, in particular from 10 to 30 or from 5 to 25, and in particular from 5 to 20) with chambers of different sizes may be used, to increase the degree of fibrillation.

Abrasion or milling treatment (see for example Iwamoto S et al., 2007 Applied Physics A89 (2): 461-66) is based on the use of a grinding device able to exert shearing forces supplied by millstones.

The pretreated cellulosic substrate, generally in the form of a cellulose pulp, is passed between a static millstone and a rotating millstone, typically rotating at a speed of the order of 1500 revolutions per minute (rpm). Several passes (generally between 2 and 5) may be necessary to obtain fibrils of nanometric size.

A device of the mixer type (for example as described in Unetani K et al., Biomacromolécules 2011, 12(2), pp. 348-53) may also be used for producing microfibrils starting from the pretreated cellulosic substrate, for example starting from a suspension of wood fibers.

The treatment of cryogrinding (or cryocrushing) (Dufresne et al., 1997, Journal of Applied Polymer Science, 64 (6): 1185-94) consists of grinding a suspension of pretreated cellulosic substrate, frozen beforehand with liquid nitrogen. The ice crystals formed inside the cells cause the cell membranes to explode and release wall fragments. These methods are generally employed for producing microcellulose fibrils from agricultural products, or from agricultural waste.

### Step (s) of Posttreatment of the Cellulosic Substrate

In certain embodiments, the process for defibrillation and/or manufacture of cellulose fibers comprises at least one step of posttreatment of the cellulosic substrate, carried out after said substrate has undergone mechanical treatment.

Generally, said at least one step of posttreatment aims to increase the degree of fibrillation of the celluloses (especially nanocelluloses) obtained and/or to endow said nanocelluloses with novel mechanical properties, depending on the applications envisaged.

Said at least one step of posttreatment may in particular be selected from an acid treatment, an enzymatic treatment, oxidation, acetylation, silylation, or else derivatization of certain chemical groups carried by the microfibrils. Reference may also be made for example to the document of Lavoine N et al. (Carbohydrate Polymers, 2012, (92): 735-64), which describes in particular (point 2.3, pages 747 to 748) post treatments that can be combined with various pretreatments and mechanical treatments of cellulose fibers.

### EXAMPLES

### Example 1: Fungal Strains and Culture Conditions

The strain of Aspergillus japonicus used in this study is held in the collection of the International Center for Microbial Resources—Filamentous Fungi (CIRM-CF., INRA, Marseille, France) under accession number CIRM BRFM. It was cultured on MYA2 agar medium, containing 20 g/l of malt extract, 20 g/l of agar and 1 g/l of yeast extract. The spores were collected and stored at −80° C. in 20% glycerol and 80% water.

The liquid culture media containing an autoclaved fraction of maize bran or beet pulp (supplied by ARD, Pomacle, France) as carbon source and protein secretion inducer were prepared as follows: 15 g/l of inducer; 2.5 g/l of maltose; 1.84 g/l of diammonium tartrate as nitrogen source; 0.5 g/l of yeast extract; 0.2 g/L of KH₂PO₄; 0.0132 g/L of CaCl₂·2H₂O and 0.5 g/L of MgSO₄·7H₂O. Another inducer medium was prepared using 4 g/l of Avicel (Sigma-Aldrich, USA); 10 g/l of xylose; 1.8 g/l of diammonium tartrate; 0.5 g/l of yeast extract; 0.2 g/l of KH₂PO₄; 0.0132 g/L of CaCl₂·2H₂O and 0.5 g/L of MgSO₄≮7H₂O.

The three culture media were inoculated with 2×10⁵ spores/mL of the five strains, and incubated in baffled flasks in the dark at 30° C. with rotary stirring at 105 rpm (Infors HT, Switzerland).

### Example 2: Preparation of the Secretomes

After incubation for 7 days, the cultures were stopped and the liquid medium was separated from the mycelium using Miracloth (Calbiochem, USA). 40 mL was filtered on 0.22 μm polyethersulfone membranes (Merck-Millipore, Germany) and then diafiltered and concentrated on polyethersulfone membranes with a cutoff threshold of 10 kDa (Vivaspin, Sartorius, Germany) in 50 mM sodium acetate buffer pH5 to a final volume of 2 mL. The secretomes were stored at −20° ° C., and their total concentration of proteins was evaluated by Bradford's methods (BioRad Protein Assay, Ivry, France) and BCA (Bicinchoninic Acid Protein Assay, Sigma-Aldrich) using a standard range of bovine serum albumin (BSA).

### Example 3: Gene Cloning and Recombinant Expression in Pichia pastoris

The selected protein sequences, from which the predicted sequences of the native signal peptides had been removed, were used for generating coding nucleotide sequences optimized for expression in P. pastoris. Complete synthesis of the genes was performed (Genewiz, USA) and they were cloned into the pPICZαA expression vector (Invitrogen), so as to be in phase with the signal sequence used (that of the α factor of yeast) and with the sequence coding for the C-terminal polyhistidine tag.

The plasmids were amplified by transformation of competent cells DH5α of Escherichia coli, then purified using a HiSpeed Plasmid Midi kit (Qiagen, The Netherlands), linearized with the PmeI restriction enzyme and transformed into X-33 cells of Pichia pastoris (Invitrogen) by electroporation. The transformants were isolated on agar medium containing Zeocin (100 and 500 mg/L).

To test the expression of each vector, several Zeocin-resistant transformants were cultured on a 24-well plate, in 5 mL of BMGY growth medium containing 0.2 g/L of biotin, at 30° C. and 250 rpm for about 16 h, until an optical density at 600 nm of between 2 and 6 was reached. After centrifugation, the cells were taken up in 1 mL of BMMY medium and incubated at 20° C. and 200 rpm for 3 days in order to induce expression of the proteins; the medium was supplemented each day with 3% (v/v) methanol. The culture supernatants were purified by affinity chromatography with Ni-NTA beads according to an automated procedure described by Haon et al. (“Recombinant protein production facility for fungal biomass-degrading enzymes using the yeast Pichia pastoris”. Front. Microbiol. 6. 2015), and the purified proteins were deposited on unstained SDS-PAGE gel with detection by photoactivation and fluorescence (BioRad, France) to verify their production.

In order to produce the proteins in sufficient quantities, the transformants having the best rate of secretion were cultured in 2 L of BMGY medium containing 1 mL/L of PTM4 (Pichia Trace Minerals 4) salts, 0.2 g/L of biotin in flasks at 30° C. and 250 rpm for about 16 h, until an optical density at 600 nm of between 2 and 6 was reached. Expression of the proteins was induced by incubating the cells in 400 mL of BMMY medium containing 1 mL/L of PTM4 salts at 20° C. and 200 rpm for 3 days, during which the medium was supplemented with 3% (v/v) methanol each day. The supernatants were recovered by centrifugation (4000×g, 4ºC, 10 min) and filtered on 0.45 μm membranes (Millipore) to remove the remaining cells. A His-Bind Resin chelated nickel column (1.9 cm³; GE Healthcare, UK) was connected to an FPLC Äkta apparatus (GE Healthcare) and equilibrated with 50 mM Tris-HCl (pH 7.8), 50 mM NaCl and 10 mM imidazole. After adjusting the pH to 7.8, the supernatants were loaded on the column at 4ºC. The column was washed with the equilibration buffer (50 mM Tris-HCl pH 7.8, 50 mM NaCl, 10 mM imidazole) and the enzymes were eluted with the same buffer containing 150 mM of imidazole. The eluates were concentrated using Amicon centrifugation units (cutoff threshold 10 kDa; Millipore) at 4000×g and washed several times with 50 mM sodium acetate buffer (pH 5).

A fraction of each eluate was loaded on unstained SDS-PAGE gel with detection by photo-activation and fluorescence (Bio Rad, France) to verify their purity. The concentration of proteins was determined by absorption at 280 nm using a Nanodrop ND-2000 spectrophotometer (Thermo Fisher Scientific) and theoretical molecular weights and coefficients calculated from the protein sequences.

### Example 4: Tests of Degradation of Cellulose in the Presence of Polypeptides According to the Invention

The tests of degradation of cellulose by the polypeptides with polysaccharide oxidase activity were carried out in triplicate, in a volume of 300 μL containing 1% (w/v) of Avicel (Sigma-Aldrich) or of PASC (Phosphoric Acid Swollen Cellulose), prepared from Avicel by the method described by Wood (“Preparation of crystalline, amorphous, and dyed cellulase substrates. In Methods in Enzymology (Academic Press), pp. 19-25) in 50 mM phosphate buffer pH 6. The LPMOs (0.5 to 5 μM) and the electron donor (L-cysteine or ascorbate, 10 μM to 1 mM) were added, in the presence or absence of H₂O₂ (100 μM), and the tubes were incubated in a thermomixer (Eppendorf, Montesson, France) at 50° C. and 850 rpm, for 4 to 16 h. The reaction was stopped by scalding the samples at 100° C. for at least 10 min, and then they were centrifuged at 15000×g for 5 min to separate the soluble products from the insoluble fraction.

To test the synergy of the polypeptides of interest with the CBHI of T. reesei, a reaction mixture with a total volume of 800 μL containing 1% (w/v) of cellulose nanofibrils in 50 mM acetate buffer pH 5.2, as well as 8 μg of LPMO and 1 mM of ascorbate was incubated in a thermomixer (Eppendorf) at 50° ° C. and 850 rpm, for 16 h. The samples (in triplicate) were scalded at 100° ° C. for at least 10 min, and then centrifuged at 15000×g for 5 min. The remaining insoluble fraction of the substrate was washed twice in the buffer, and hydrolysis with the CBHI from T. reesei (0.8 μg; supplied by IFPEN) was carried out in a volume of 800 μL containing 50 mM acetate buffer pH 5.2, for 30 min at 50° C. and 850 rpm. The reaction was stopped as described above, and the soluble and insoluble fractions were separated.

The mono- and oligosaccharides resulting from the degradation of the cellulose substrates were detected by HPAEC-PAD (Dionex, Thermo Fisher Scientific), by the method described by Westereng et al. (2013), using unoxidized cello-oligosaccharides (DP2 to DP6) as standards (Megazyme, Ireland).

### Example 5: Substrates and Enzyme Cocktails

Pretreated wheat straw, miscanthus and poplar were obtained from IFP Energies Nouvelles (Rueil-Malmaison, France). The biomasses were pretreated by steam explosion in acid conditions, washed with hot water to remove the free products, and dried at 55° C. After one week at room temperature, they were ground and sieved, only retaining the particles smaller than 0.8 mm, and their water content (% w/w) was determined by drying at 105° C. in a stove with natural convection (VWR, USA).

The “K975” cocktail consists of the secretome of the CL847 strain of Trichoderma reesei, produced in the presence of lactose according to the protocol detailed in Herpoël-Gimbert et al. (Comparative secretome analyses of two Trichoderma reesei RUT-C30 and CL847 hypersecretory strains, Biotechnology for Biofuels 2008, 1:18), the specific β-glucosidase activity of which is 0.8 IU/mg. It was supplemented with a commercial cocktail of β-glucosidases from Aspergillus niger SP188 (Novozyme, Denmark).

### Example 6: Enzymatic Hydrolyses

The enzymatic hydrolyses in 2-mL tubes were carried out in triplicate in a reaction volume of 1 mL, in the presence of wheat straw, miscanthus or poplar (50 mg of dry biomass), in 50 mM sodium acetate buffer (pH 4.8), to which chloramphenicol (0.1 g/L) was added to prevent microbial contamination. This mixture was incubated for at least 1 h at 45° C. with rotary stirring at 850 rpm (Infors) in order to impregnate the biomass, then the K975 cocktail was added at a rate of 5 mg/g of dry matter (DM), and the SP188 cocktail was added in amounts to give total β-glucosidase activity of 80 IU/g DM.

For the supplementation tests, 10 μL of secretomes was added (or 10 μl of buffer in the reference conditions). Hydrolysis was carried out at 45° C. with rotary stirring at 850 rpm (Infors). At each sampling time, the triplicates were scalded at 100° C. for at least 5 min to inactivate the enzymes, then cooled and centrifuged at 15000×g for 5 min, and the supernatant was withdrawn and stored at −20° C.

The miniaturized enzymatic hydrolyses were carried out on 96-well microplates, in which 100 μL of suspension of biomass at 6.3% (w/v) in 50 mM sodium acetate buffer (pH 4.8) in the presence of chloramphenicol (0.1 g/L) was distributed. During distribution, the biomass suspension was taken using a multichannel pipette in a beaker with continuous magnetic stirring, and then the plates were sealed and stored at −20° C.

For the supplementation tests, the enzymes were distributed by means of a Tecan Genesis Evo 200 robot (Tecan, Lyon, France): the K975 and SP188 cocktails were distributed in the amounts indicated above, in a volume of 15 μL, and then 10 μL of secretomes diluted 10× was added (or 10 μL of buffer in the reference conditions, present on each plate). Each test was performed in septuplicate, to which a control well is added containing the enzymes only (without biomass); one column of each plate was dedicated to the control conditions containing the biomass only (without enzymes). During hydrolysis, the sealed plates were incubated at 45° C. with rotary stirring at 850 rpm (Infors), for 24 to 96 h. At each sampling time, the corresponding plates were centrifuged after adding 120 μL of buffer, then the supernatant was filtered and stored at −20° C.

For both types of hydrolyses (“sequential” or “simultaneous”), the glucose concentration in the samples was measured using the Glucose GOD-PAP reagent (Biolabo, Maizy, France) using a standard glucose range, and the yields were calculated taking into account the amount of cellulosic glucose initially present.

Several hydrolyses were carried out for each condition, and so as to be able to combine the results, the yields obtained in the presence of secretomes were translated into percentage improvement relative to the internal reference of each hydrolysis. A Student t test was carried out for each condition to determine whether the mean value of the results was statistically different than the mean value of the references, using the p-value as a criterion.

### Example 7: Tests of Saccharification of Biomasses with T. reesei Supplementation

In order to determine whether the secretomes produced may be of interest for improving yields in enzymatic hydrolysis, they were tested in conditions approaching those envisaged in the industrial processes for bioconversion of lignocellulosic biomass. Three model biomasses, with different origin and compositions, were selected for this study: wheat straw, miscanthus and poplar. These substrates, derived from agricultural waste and from dedicated energy crops, are often envisaged as raw materials for bioethanol production in Europe. They were pretreated by steam explosion in the presence of dilute sulfuric acid (see Table 1). After washing and drying, their composition of sugars was determined by GC after acid hydrolysis.

TABLE 1
Pretreatment conditions and composition of
the three model substrates in this study

| | Poplar | Miscanthus | Straw |
|---|---|---|---|
| Steam temperature (° C.) | 195 | 180/190 | 195 |
| Pressure (bar) | 15 | 10.5/13 | 15 |
| Pretreatment time (min) | 7.5 | 7.5 | 2 |
| [H₂SO₄] | 0.7% | 0.4% | 0.7% |
| Composition (% dry weight) | | | |
| Rhamnose | 0.27 | 0.44 | 0.15 |
| Arabinose | 0.11 | 0.39 | 0.52 |
| Xylose | 0.63 | 2.09 | 1.58 |
| Mannose | 0.18 | 0 | 0.16 |
| Total glucose | 53.28 | 59.43 | 53.92 |
| Cellulosic glucose | 50.85 | 57.01 | 51.30 |
| Water content (% w/w) | 6.26% | 5.66% | 6.97% |

The cellulolytic cocktail from T. reesei used (designated K975) is derived from the CL847 strain, cultured in the presence of lactose; as the latter has rather low β-glucosidase activity, a commercial preparation of β-glucosidases from A. niger (SP188, Novozyme) was added to it in excess, in order to ensure that the potential improvements in performance on supplementation are not due to a simple increase in β-glucosidase activity. This reference cocktail was used for carrying out hydrolysis of the three biomasses selected, with a relatively high concentration of biomass (5% w/v) in order to keep close to realistic conditions from the standpoint of the method, and a low enzyme dose (5 mg/g of dry matter), corresponding for industry to conditions making it possible to reduce costs, and for which the margin of improvement of hydrolysis yields is high.

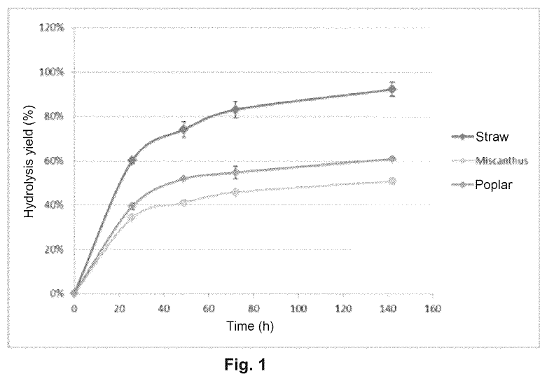

The kinetics of this hydrolysis over 5 days is presented in FIG. 1. In the chosen conditions, the final hydrolysis yield of wheat straw is close to 100%, which means that nearly all of the cellulose has been transformed into glucose monomers. Miscanthus and poplar are more stubborn, since in the same conditions they are only hydrolyzed to 50 and 60% respectively. The objectives of improvement are multiple: in the case of straw, it will be a question of increasing the yields at the start of hydrolysis (at 24 and 48 h) and therefore accelerating the reaction, whereas for miscanthus and poplar it will be possible to improve both the initial kinetics and the yield after a longer period (96 h for example).

In the same conditions, the reference cocktail was then supplemented with the various secretomes from Aspergillus. For the sake of experimental simplification, equal volumes of each of the concentrated secretomes were added to a fixed amount of cocktail, according to the protocol established in Berrin et al. (“Exploring the Natural Fungal Biodiversity of Tropical and Temperate Forests toward improvement of Biomass Conversion. Appl. Environ. Microbiol. 78, 6483-6490, 2012). The amount of proteins added is not the same in all the samples, but it reflects the initial proportions of proteins secreted by the fungal strains on each culture medium. Initially these tests were performed in tubes, in triplicate, but as the biomasses used are heterogeneous, it was found that there was considerable variability of the results. The saccharification tests were miniaturized on microplates and automated using a Tecan robot, according to a method already developed in Navarro et al. (“Automated assay for screening the enzymatic release of reducing sugars from micronized biomass. Microb. Cell Factories 9, 58, 2010).

Thus, the biomass is ground finely and a sufficiently homogeneous suspension is prepared so that it can be pipetted onto microplates, before adding a reference cocktail and various secretomes. Each assay has 7 replicates. The data obtained from three hydrolysis series (one in tubes and two on microplates) were analyzed statistically using a Student test.

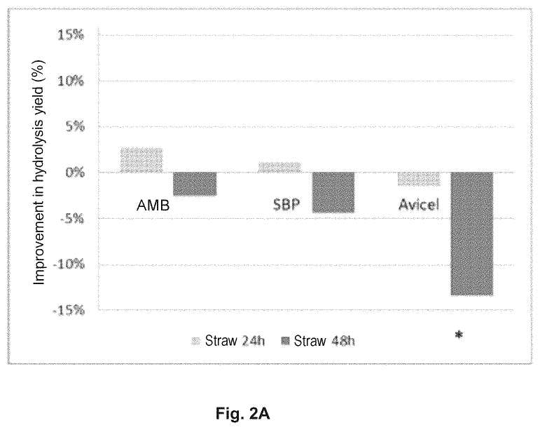

With the data derived from the saccharification test that had been developed, the influence of the secretomes on the hydrolysis yield can be compared for each biomass (FIG. 2A-C). There are large differences in performance on the three biomasses: several secretomes make it possible to improve both the hydrolysis of straw and that of miscanthus, starting from 24 h.

### Example 8: Saccharification Tests of Biomass in the Presence of Polypeptides with Polysaccharide Oxidase Activity

The supplementation tests of the cocktail from T. reesei with the polypeptides for saccharification of miscanthus were carried out in triplicate by the method of enzymatic hydrolysis described above, using 5 mg of biomass, 1 mg/g DM of the K975 cocktail and 80 IU/g DM of β-glucosidase supplied by the SP188 cocktail, for 24 h at 45° C. and 850 rpm. In some of the samples, treatment with the LPMOs (2.2 μM) in the presence of 1 mM ascorbate was carried out for 24 h before the start of hydrolysis; in the others, the LPMOs and the ascorbate were added at the start of hydrolysis, at the same time as the cellulolytic cocktail.

The polypeptides were not added to the reference samples. After 24 h, the reaction was stopped and the glucose concentration in the samples was measured using the Glucose GOD-POD reagent (Biolabo).

### Example 9: Selection and Production of Fungal Proteins of Interest

So as to be able to study the function of the polypeptides of interest, it was decided to produce them heterologously in the yeast Pichia pastoris, by means of synthetic genes placed in a vector. Several versions of the sequence of Aspergillus aculeatus were expressed (with the N-terminal X273 module only, with a truncated C-terminal extension, and with the complete C-terminal extension), as well as a similar protein comprising a very short C-terminal extension, found in Podospora anserina (PaX273).

Each of the constructs tested begins with a signal peptide, followed by the n-terminal histidine involved in the catalytic triad, and ends at a variable terminal position. For the clones derived from Aspergillus aculeatus, the reference polypeptide sequence is SEQ ID No. 2. For the clone derived from Podospora anserina, the reference polypeptide sequence is SEQ ID No. 1.

TABLE 2
Polypeptide sequences selected for expression in Pichia pastoris

| | | Size | Terminal |
|---|---|---|---|
| | Source organism | (kDa) | position |
| AaX273_complete | Aspergillus | 30.2 | 314 |
| | aculeatus | | |
| AaX273_truncated | Aspergillus | 19.6 | 200 |
| | aculeatus | | |
| AaX273_Nter | Aspergillus | 19.0 | 193 |
| | aculeatus | | |
| PaX273 | Podospora | 20.7 | 209 |
| | anserina | | |

In total, 4 genes were therefore synthesized and transformed into Pichia pastoris (see Table 2). The corresponding transformants were cultured in small volumes, according to the accelerated procedure elaborated in the laboratory (Haon et al., “Recombinant protein production facility for fungal biomass-degrading enzymes using the yeast Pichia pastoris. Front Microbiol. 6, 2015) in order to verify proper expression of the proteins.

### Example 10: Investigation of a New Family of Fungal LPMOs

### Bioinformatic Analysis of the X273 Family

In order to measure the extent of the X273 family in the fungal genomes, a homology search based on the sequence of the X273 module of Podospora anserina, with a length of 165 amino acids, was carried out, using the NCBI Blast tool, and only keeping the closest sequences (e-value less than or equal to 1e⁻¹⁸).

Secondly, in order to optimize selection of the polypeptide sequences of interest, a sequence alignment carried out using Clustal Omega (EMBL-EBI) is taken as a basis, then curating manually in order to discard the following sequences:
N-ter or C-ter fragments not corresponding to whole proteins (in particular those not comprising the 1st histidine, characteristic of the LPMOs);
Sequences whose N-ter end does not correspond to a signal peptide according to the SignalP 4.1. software;
Sequences comprising important insertions or deletions, possibly due to poor splicing models;
Sequences having a very long C-terminal extension.

Besides SEQ ID No. 1 and 2, we thus get a refined list of 379 polypeptide sequences (SEQ ID No. 7 to 383), derived from fungal organisms, and mainly ascomycetes: 335 sequences, versus 41 derived from basidiomycetes and 3 from unclassified fungi.

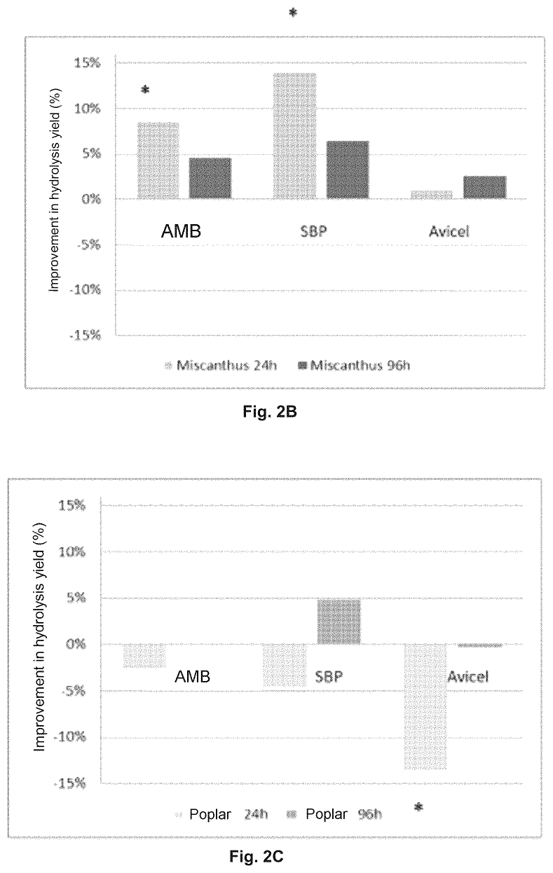

The consensus amino acids are shown graphically in FIG. 3, after alignment of the 378 catalytic modules corresponding to the protein sequences SEQ ID No. 1, 2 and 7 to 382. The figure was generated by the WebLogo application, according to Crooks et al. (WebLogo: A Sequence Logo Generator. Genome Res. 14, 1188-1190; 2004).

The alignment of the sequences corresponding to the X273 module reveals several well conserved regions within the family. All the sequences comprise in particular an N-terminal histidine, as well as a second strictly conserved histidine, which is a characteristic of the active center (“Histidine brace”) of all the LPMOs characterized to date. There are about twenty other strictly conserved residues, including 6 cysteines potentially involved in the formation of disulfide bridges. In addition to the X273 module, most of the proteins in the list possess a C-terminal extension, of variable length depending on the species. For 7 of them, found in 4 different organisms, this extension comprises a cellulose binding module (CBM1).

In Table 3, the relative positions of these conserved amino acids are determined relative to the reference sequences SEQ ID No. 1 and SEQ ID No. 2.

TABLE 3
Conserved positions in the X273 catalytic module

| Conserved amino acids | Position in SEQ ID 1 | Position in SEQ ID 2 |
|---|---|---|
| Histidine (H) | 19, 105 | 20, 109 |
| Glycine (G) | 31, 107 | 32, 111 |
| Proline (P) | 25 | 26 |
| Arginine (R) | 28 | 29 |
| Cysteine (C) | 38, 69, 74, 157, | 39, 73, 78, 162, |
| | 163, 179 | 168, 186 |
| Asparagine (N) | 110 | 114 |
| Glutamine (Q) | 166, 174 | 171, 181 |
| Tryptophan (W) | 169 | 174 |
| Glycine (D) or | 20, 76 | 21, 80 |
| Alanine (A) | | |
| Glycine (D) or | 161 | 166 |
| Asparagine (N) | | |
| Aspartic acid (D) or | 49 | 50 |
| Asparagine (N) | | |
| Proline (P) or | 151 | 155 |
| Alanine (A) | | |
| Threonine (T) or | 175 | 182 |
| Asparagine (N) | | |
| Tyrosine (Y) or | 176 | 183 |
| Phenylalanine (F) | | |
| Aspartic acid (D) or | 181 | 188 |
| Leucine (L) | | |

### Heterologous Production and Activity Assay

The clones of P. pastoris expressing the X273 of A. aculeatus and of P. anserina were used for producing the proteins in 1 L flasks. The version of the protein of A. aculeatus retained is the one that was produced most in small volumes, i.e. the one that was truncated by leaving out about ten amino acids of the C-terminal extension. After purification, a total amount of 47.5 mg is obtained for AaX273 and 61 mg for PaX273. The purified proteins were then loaded with copper, so that their active center is functional, and the excess copper was removed by diafiltration. An analysis by inductively-coupled plasma mass spectrometry (ICP-MS) confirmed the presence of about one copper atom per molecule of X273.

Using a fluorimetric assay in the presence of Amplex Red, production of ELCL by the X273 enzymes in the presence of an electron donor (ascorbic acid) and in the absence of substrate could be established, which indicates that these enzymes have an oxidation activity and behave like the other families of LPMOs.

### Characterization of the Degradation Products of Cellulose

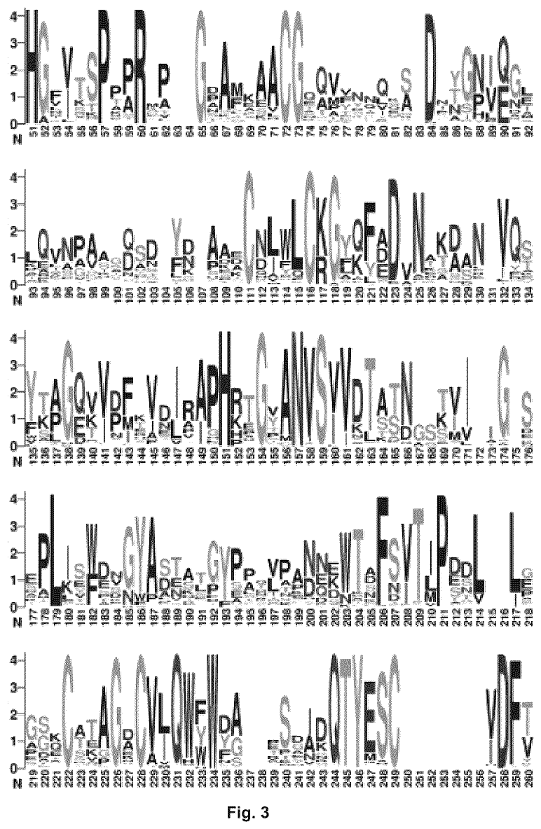

In order to determine whether the LPMOs have an activity on cellulose, their effect can be observed indirectly by utilizing their synergy with cellulases. Thus, after leaving the X273 proteins to act on cellulose nanofibrils (CNFs), the latter were hydrolyzed with CBHI (Ce17A) from T. reesei. The amount of cellobiose measured by ion exchange chromatography (HPAEC-PAD) at the end of this hydrolysis is greater in the samples that had been treated with X273 than in the control sample (see FIG. 4).

This indicates that the X273 seem to introduce cuts in the cellulose chains, which serve as new ends for attack by cellobiohydrolase, which therefore releases more cellobiose than when it acts alone.

Visualization of the direct action of the LPMOs on cellulose fibers is more difficult, since only the soluble polysaccharides (the degree of polymerization of which is generally under 6) are detected by chromatographic methods. However, it is possible to quantify the short cellulose chains released from the accumulation of these cleavages, and determine whether they are native or oxidized oligosaccharides (in particular of C₁ and/or C₄).

Thus, assays were conducted on PASC (Phosphoric Acid Swollen Cellulose) and Avicel (microcrystalline cellulose), in the presence of H₂O₂, and decreasing the amount of electron donor according to the conditions proposed by Bissaro et al. (“Oxidative cleavage of polysaccharides by monocopper enzymes depends on H₂O₂”. Nat. Chem. Biol. 13, 1123-1128; 2017). By increasing the amounts of AaX273 enzyme, products could be observed on both substrates. The two enzymes release cellobiose, cellotriose, cellotetraose and cellopentaose.

### Saccharification of Pretreated Biomass

After estimating the activity of f the X273 on cellulose, their activity on more complex substrates was evaluated.

As the two secretomes containing X273 had shown an improvement of hydrolysis on pretreated miscanthus, this biomass was chosen for carrying out the saccharification assays. To facilitate homogenization, a smaller proportion of biomass was used than previously (0.5% w/v), and the enzyme dose of the reference cocktail was also decreased (1 mg/g of dry matter). A dose of X273 of 0.2 mg/g of dry matter was added. Two types of hydrolysis were carried out:
by contacting the X273 proteins with the biomass for 24 h and then adding the reference cocktail; and
by allowing said proteins and the reference cocktail to act simultaneously.

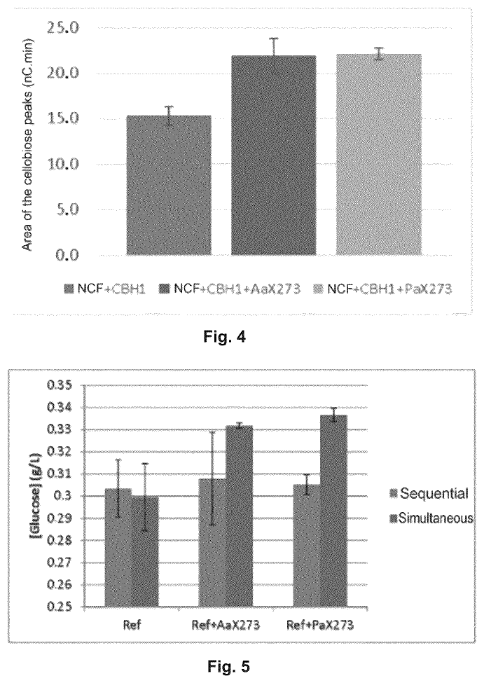

The data obtained after 24 h of hydrolysis of miscanthus with the reference cocktail, with or without addition of AaX273 or PaX273, and in particular of AaX273, are illustrated in FIG. 5.

Thus, after 24 h of hydrolysis, an increase in the amount of glucose is observed in the presence of the LPMOs, mainly when the proteins are added simultaneously. There is therefore a synergy between the X273 and the cellulolytic cocktail from T. reesei.

### Example 11: Pretreatment of Cellulosic Substrate PASC in the Presence of a Selection of Enzymes Belonging to the X273 Family

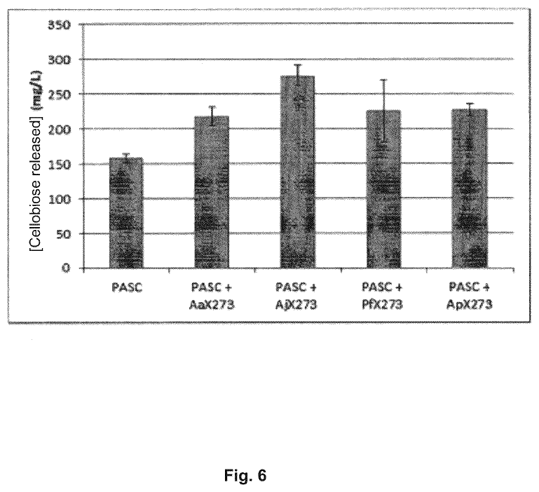

The results obtained are shown in FIG. 6, according to a protocol similar to that used for FIG. 4. Four enzymes were tested, and their effect on the level of cellobiose released in the presence of a cellulase from Trichoderma reesei was compared with a PASC control without prior contacting with said enzyme.

It can thus be seen that all of these enzymes tested, of sequence SEQ ID No. 2, SEQ ID No. 175, SEQ ID No. 208, and SEQ ID No. 383, improve the accessibility of the cellulose for the cellulases, relative to a simple PASC treatment, by improving the cellobiose released after treatment with a cellulase from T. reesei (synergistic effect).

For reference, the sequence SEQ ID No. 383 has at least 40% sequence identity with SEQ ID No. 1 and SEQ ID No. 2.

### SEQUENCE LISTING

| SEQ ID | General Description |
|---|---|
| 1 | Reference polypeptide sequence of enzyme with polysaccharide |
| | oxidase activity derived from Podospora anserina |
| | (referenced PaX273) |
| 2 | Reference polypeptide sequence of enzyme with polysaccharide |
| | oxidase activity derived from Aspergillus aculeatus |
| | (referenced AaX273) |
| 3 | Signal peptide of Pax273 |
| 4 | Signal peptide of AaX273 |
| 5 | Catalytic module of Pax273 |
| 6 | Catalytic module of AaX273 |
| 7-383 | Other polypeptide sequences related to the X273 family |

SEQ ID NO. 1
MHFSTVSSALGLASLVSAHQVVLKPASRKPQDATTEACQRAMVNFYKQDE
TSYPEAFLRSNPLPDRNKCNLFLCKGYQFADNAANVQSYKPGDSVEYEVY
IRIPHSGYANYSIVDTTINKVLQSPLVSWASQYAASKPPADQTKESVKIP
ELGAQCAAAGVCVLQWHWFGAGQTYQSCTDFTVAPAEPAPEHGHGHRIRQ
QSRW
SEQ ID NO. 2
MKQTGSILALAGLVSMANAHGEVTSPQPRMPGSAMEKACGQQVYNNQEAD
NYGNIQGELQIASGQSDYDAEACDIWLCKGYKYADNTANVQSYKPGEVID
FTVDIRAPHTGTANVSVVDTATNTMLSQPLIYWSVYASTATGVTANETSF
SVTMPTDLGDKCNEAGACVLQWWWDARSIDQTYESCVDFTLSGSSSSSSS
SSSSSSSSSSSSSSSSSSAAAAVATSAASSSTSSAAETTTAASAAVTSPA
AANNIAPVSSSGPTTLATSVKQTATAAAVSSATSTSVSLPTDGTAEEQLT
WVASVFKALLNYAN
SEQ ID NO. 3
MHFSTVSSALGLASLVSA
SEQ ID NO. 4
MKQTGSILALAGLYSMANA
SEQ ID NO. 5
HGVVLKPASRKPQDATTEACGRAMVNFYKQDETSYPEAFLRSNPLPDRNK
CNLFLCKQYQFADNAANVQSYKPQDSVEYEVYIRIPHSGYANVSIVDTTT
NKYLGSPLVSWASGYAASSKPPADQTKFSVKIPELGAQCAAAGVCVLQWH
WFGAGQTYQSCTDFT
SEQ ID NO. 6
HQFVTSPQPRMPGSAMEKACGQQVYNNQEADNYGNIQGELQIASGQSDYD
AEACDIWLCKGYKYADNTANVQSYKPQEVIDFTVDIRAPHTGTANVSVVD
TATNTMLSQPLIYWSVYASTATQVTANETSFSVTMPTDLGDKCNEAGACV
LQWWWDARSIDQTYESCVDFT

1. An isolated polypeptide with polysaccharide oxidase activity, comprising a sequence having an amino acid identity of at least 20% with a reference polypeptide sequence SEQ ID No. 2 or SEQ ID No. 1, using a BLAST-P comparison method, said BLAST-P comparison method giving an e-value of 1e⁻¹⁸ or less.

2. The isolated polypeptide with polysaccharide oxidase activity as claimed in claim 1, the sequence having an amino acid identity of at least 40% with the reference polypeptide sequence SEQ ID No. 2 or SEQ ID No. 1, said BLAST-P comparison method giving an e-value of 1e⁻¹⁸ or less.

3. The isolated polypeptide with polysaccharide oxidase activity as claimed in claim 1, the sequence having an amino acid identity of at least 80% with the reference polypeptide sequence SEQ ID No. 2 or SEQ ID No. 1, said BLAST-P comparison method giving an e-value of 1e⁻¹⁸ or less.

4. The isolated polypeptide with polysaccharide oxidase activity as claimed in claim 1, comprising a reference polypeptide sequence selected from SEQ ID No. 5 to SEQ ID No. 383.

5. A composition with polysaccharide oxidase activity, comprising a polypeptide with polysaccharide oxidase activity as claimed in claim 1.

6. The composition with polysaccharide oxidase activity as claimed in claim 5, further comprising at least one polypeptide with polysaccharide degradase activity selected from: a cellulase, a hemicellulase, a ligninase and a carbohydrate oxidase.

7. A kit comprising at least:
a first part comprising a polypeptide with polysaccharide oxidase activity as claimed in claim 1, or a composition comprising said polypeptide with polysaccharide oxidase activity;
a second part comprising a polypeptide with polysaccharide degradase activity selected from a cellulase, a hemicellulase, a ligninase and a carbohydrate oxidase, or a composition comprising said polypeptide with polysaccharide degradase activity.

8. A host able to express recombinantly a polypeptide with polysaccharide oxidase activity as claimed in claim 1.

9. An isolated nucleic acid coding for a polypeptide with polysaccharide oxidase activity as claimed in claim 1.

10. A method of obtaining a sugar starting from a polysaccharide substrate, comprising at least one step of contacting said substrate with a polypeptide with polysaccharide oxidase activity as claimed in claim 1.

11. A method for producing alcohol starting from a lignocellulosic substrate, comprising obtaining a sugar by a method as claimed in claim 10, and the alcoholic fermentation of said sugar by an alcohol-producing microorganism.

12. A method for preparing a cellulosic substrate for producing cellulose fibers, said method comprising at least the following steps consisting of:
a) providing a cellulosic substrate able to form cellulose fibers;
b) in the presence of an electron donor, contacting said substrate with at least one polypeptide with polysaccharide oxidase activity in conditions suitable for ensuring oxidative cleavage of said cellulose fibers;
wherein said polypeptide with polysaccharide oxidase activity has an amino acid identity of at least 20% with a reference polypeptide sequence SEQ ID No. 2 or SEQ ID No. 1, using a BLAST-P comparison method, said BLAST-P comparison method giving an e-value of 1e⁻¹⁸ or less.

13. A method of defibrillating a cellulosic substrate, said method comprising at least the following steps consisting of:
a) providing a cellulosic substrate able to form cellulose fibers, which had been brought into contact beforehand with an electron donor and a polypeptide with polysaccharide oxidase activity, in conditions suitable for ensuring oxidative cleavage of said cellulose fibers in the presence of an electron donor; and
b) treating said cellulosic substrate mechanically, in order to defibrillate said substrate,
wherein said polypeptide with polysaccharide oxidase activity has an amino acid identity of at least 20% with a reference polypeptide sequence SEQ ID No. 2 or SEQ ID No. 1, using a BLAST-P comparison method, said BLAST-P comparison method giving an e-value of 1e⁻¹⁸ or less.

14. A method of producing cellulose fibers, said method comprising at least the following steps consisting of:
a) providing a cellulosic substrate able to form cellulose fibers;
b) in the presence of an electron donor, contacting said cellulosic substrate with at least one polypeptide with polysaccharide oxidase activity in conditions suitable for ensuring oxidative cleavage of said cellulose fibers;
c) treating said cellulosic substrate mechanically, in order to produce said cellulose fibers starting from said cellulosic substrate,
wherein said polypeptide with polysaccharide oxidase activity has an amino acid identity of at least 20% with a reference polypeptide sequence SEQ ID No. 2 or SEQ ID No. 1, using a BLAST-P comparison method, said BLAST-P comparison method giving an e-value of 1e⁻¹⁸ or less.

15. Cellulose fibers derived from a method of defibrillating a cellulosic substrate as claimed in claim 13.

16. A method of obtaining a sugar starting from a polysaccharide substrate, comprising at least one step of contacting said substrate with a composition with polysaccharide oxidase activity as claimed in claim 5.

17. Cellulose fibers derived from a method of producing cellulose fibers as claimed in claim 14.
